(19) United States
(12) Patent Application Publication
(10) Pub. No.: US 2013/0143746 A1
(43) Pub. Date: Jun. 6, 2013

(54) METHOD FOR DETECTING GENE REGION FEATURES BASED ON INTER-ALU POLYMERASE CHAIN REACTION

(71) Applicant: PharmacoGenetics Ltd., Hong Kong (CN)

(72) Inventors: Hong Xue, Hong Kong (CN); Lingling Mei, Hong Kong (CN)

(73) Assignee: PHARMACOGENETICS LTD., Hong Kong (CN)

(21) Appl. No.: 13/678,693

(22) Filed: Nov. 16, 2012

Related U.S. Application Data
(63) Continuation-in-part of application No. 13/637,444, filed on Nov. 1, 2012.

(30) Foreign Application Priority Data
Mar. 30, 2010 (CN) 201010139483.5

(51) Int. Cl. G06F 19/18; C12N 15/10

(52) U.S. Cl. CPC G06F 19/18; C12N 15/1093
USPC 506/2; 506/26

(57) ABSTRACT

An array of inter-Alu gene-enriched amplicons produced by a polymerase chain reaction (“PCR”) process. The PCR process comprises combining one or a plurality of Head-type/Tail-type Alu- or AluY- or any other Alu-subfamily-consensus sequence-based primer; a genomic DNA template isolated from cells; and a PCR-extension mix. The combination of primers, DNA template; and PCR extension mix comprises an inter-Alu-PCR-mixture. After making the inter-Alu-PCR mixture, an inter-Alu PCR cycle program is used in connection with a PCR machine for a period of time to produce the array of inter-Alu gene-enriched amplicons that are sequenced by massively parallel sequencing to allow genome wide scanning of sequence and structure variations in the human genome.

### RELATED APPLICATIONS

This application is a continuation-in-part of U.S. National Phase application Ser. No. 13/637,4444, filed Sep. 26, 2012, entitled “METHOD FOR DETECTING GENE REGION FEATURES BASED ON INTER-ALU POLYMERASE CHAIN REACTION,” which application claims priority to PCT Application No. PCT/CN2011/072204, filed Mar. 28, 2011, the entire content of each of which is hereby incorporated by reference.

### FEDERALLY SPONSORED RESEARCH

Not Applicable.

### JOINT RESEARCH AGREEMENTS

Not Applicable.

### SEQUENCE LISTING

Incorporated by reference in its entirety herein is a computer-readable nucleotide sequence listing submitted concurrently herewith and having the following listed sequences:

(SEQ ID 1, or AluY278T18)
5′ GAGCGAGACT CCGTCTCA 3′
(SEQ ID 2, or AluY66H21)
5′ TGGTCTCGAT CTCCTGACCT C 3′
(SEQ ID 3, or AluT-T)
5′ AGGCTGAGGC AGGAGAATG 3′
(SEQ ID 4, or CH11)
5′ TTTAATAAAA A 3′
(SEQ ID 5, or CT11)
5′ CTATAATCCC A 3′
(SEQ ID 6, R12A/267)
5′ AGCGAGACTC CG 3′
(SEQ ID 7, or AluJo56H16)
5′ GGCTCAAGCG ATCCTC 3′
(SEQ ID 8, or A1uJo232T16)
5′ TATGATCGTG CCACTG 3′
(SEQ ID 9, or AluSq56H16)
5′ ACCTCAGGTG ATCCAC 3′
(SEQ ID 10, or AluSq263T16)
5′ AACAAGAGCG AAACTC 3′

### BACKGROUND

This invention relates to a method of genome wide scanning of sequeance and structure variations in a genome. Specifically, it relates to the detection of single nucleotide polymorphisms (“SNP”), point mutations, sequence insertion/deletions (indel) and the level of DNA CpG loci methylation, and other variations in genomic regions. The method uses the consensus sequences of Alu family, especially the AluY subfamily, to design oligonucleotide primers for genomic DNA amplification. Since the amplicons are generated by inter-Alu PCR, they are enriched with genic sequences from the genome on account of the enrichment of Alu elements in genic regions. This invention enables the preferential pre-sequencing capture of said genic sequences, using greatly reduced amounts of DNA sample. The resultant inter-Alu amplicon arrays are coupled to massively-parallel sequencing analysis to screen for genomic variations.

One aspect of this invention combines the use of a plurality of Alu consensus sequence-based PCR primers in the inter-Alu PCR amplification mixture and next generation DNA sequencing for analyzing the PCR amplicons obtained covering a significant portion of the human genome for identifying genetic variations. More specifically, a multitudinous number of amplified inter-Alu sequences are obtained using one or preferably a number of Alu consensus sequence-based PCR primers to provide a huge array of unique inter-Alu amplicons that encompass significant segments of the human genome, which when coupled with massively parallel next generation sequencing will make possible a comprehensive analysis of sequence and structure variations in a wide spectrum of sequence segments in the human genome. This novel method of capturing and sequencing a significant subset of the sequences in the human genome, based on the proximity of such sequences to widely distributed Alu elements, requires less starting genetic materials, less primers, less overall reagents, less computational workload time, and costs much less than conventional whole genome sequencing (“WGS”) protocols.

Genomic Variations.

A transposable element (“TE”) is a DNA sequence that can change its relative position (self-transpose) within the genome of a single cell. The mechanism of transposition can be either “copy and paste” or “cut and paste”. Transposition can create phenotypically significant mutations and alter the cell's genome size. The most common form of transposable element in humans is the Alu sequence. Alu-sequences are a family of repetitive elements in the human genome. Generally, Alu elements are about 300 base pairs long and are therefore classified as short interspersed elements (“SINEs”) among the class of repetitive DNA elements. There are between 300,000 and a million replications of Alu sequences in the human genome. It has been estimated that about 10.7% of the human genome consists of Alu sequences. However less than 0.5% of these elements are polymorphic.

The typical structure of an Alu sequence is 5′ Part A-Part B-PolyA Tail-3′, where Part A and Part B are similar nucleotide sequences. Expressed another way, it is believed that modern human Alu elements emerged from a head to tail fusion of two distinct fossil antique monomers over 100 million years ago, hence its dimeric structure of two similar but distinct monomers (left and right arms) joined by an A-rich linker. The length of the poly-A tail varies between Alu-families.

Alu elements were first discovered to be split in two major subfamilies known as AluJ and AluS, and other Alu subfamilies were soon discovered. Eventually a sub-subfamily of AluS that included active Alu elements was given a separate name AluY. The discovery of Alu subfamilies led to the hypothesis of master/source genes, and provided the definitive link between Transposable Elements (“TE”) (active elements) and interspersed repetitive DNA (mutated copies of active elements).

Transposable elements (“TE”) make up a large fraction of the C-value of eukaryotic cells. “C-value” means the “constant” (or “characteristic”) value of haploid DNA content per nucleus, typically measured in picograms (1 picogram is roughly 1 gigabase) and is often considered “junk DNA”. In some unique genetic systems, TE's play a critical role in development. TE's are also very useful to researchers as a means to alter DNA inside a living organism, which gives rise to altered genotypes and phenotypes.

Genotype/Phenotype

The concept of the phenotype has hidden subtleties. More specifically, it may appear that anything dependent on the genotype is a phenotype, including molecules such as RNA and proteins. Most molecules and structures coded by the genetic material are not visible in the appearance of an organism, yet they are observable (for example by Western blotting, or cell morphology) and are thus part of the phenotype. Human blood groups are an example of how the phenotype concept can be expressed at the molecular and cellular levels. In any case, the term phenotype includes traits or characteristics that can be made visible by some technical procedure. Another extension adds behavior to the phenotype, since behaviors are also observable characteristics. Often, the term “phenotype” is incorrectly used as a shorthand to indicate phenotypical changes observed in mutated organisms, such as knockout mice. As such, this specification utilizes the term “trait” to distinguish between two cell types having a difference.

Sequencing Technology.

Massively-parallel sequencing technologies (i.e. Next-generation) have transformed the landscape of genetics through their ability to produce giga-bases of sequence information in a single run. This technology advancement has cut down the cost of whole-genome sequencing and facilitated the study on disease etiologies. It has been widely employed for disease-association studies including cancer and psychiatric disorders. However, its demand for large amounts of DNA sample remains a major drawback. In most instances the use of even 3 micrograms of genomic DNA for analysis would still fall short of the stringent requirements of whole-genome sequencing, giving useful data on some genomic regions only and missing out on other regions. From the Human Genome Project, it is known that protein encoding regions and whole genic regions only account for 1% and 25% of the human genome respectively. In terms of costs, Whole Genome Sequencing (“WGS”) yields only very limited amounts of useful disease-association sequence data for genetic studies. As such, the current WGS DNA sequencing methodologies have an imbalance between high cost of sequencing, large DNA sample requirement, and limited return on investment in terms of useful data.

In view of this, novel methods are needed to selectively extract and sequence the “most-useful” portions or subsets of genomic DNA for analysis. More specifically, there is a need in the art to reduce the amount of sample DNA required to produce high quality and relevant sequence data at reduced cost. For example, the Polymerase Chain Reaction (“PCR”) technique that was developed in the late 1980's allowed a user to take a limited amount of starting genomic DNA and amplify enough DNA from any localized region in the genome to enable sequencing of that localized region. PCR was a paradigm-shift that allowed the whole genomes to be sequenced accurately.

Increasing the amount of DNA in specific regions of a genome can be achieved by means of exponential amplification through Polymerase Chain Reaction (“PCR”). However, the amount of data obtainable from PCR amplification targeting only one or a few specific genic regions is limited. Additionally, PCR employing a multiplicity of primer pairs incurs high primer cost. In this regard, U.S. Pat. Nos. 5,773,649 and 7,537,889 describe the use of inter-Alu PCR for the amplification of multiple regions in the human genome.

U.S. Pat. No. 5,773,649 titled “DNA markers to detect cancer cells expressing a mutator phenotype and method of diagnosis of cancer cell,” having Sinnett, et al. listed as inventors was issued on Jun. 30, 1998 (“the Sinnett '649 patent”) and the entire content is hereby incorporated by reference. The Sinnett '649 patent used paired primer inter-Alu PCR followed by hybridization with a probe corresponding to an instability prone locus and subjecting the amplified fragments to electrophoretic fractionation on a polyacrylamide gel to determine the presence of a variation in band profile between tumor and tumor-free DNA. However, the Sinett '649 patent only used paired primer PCR system, which yielded only limited banded patterns of PCR products that can be visualized on a gel electrophoretogram.

U.S. Pat. No. 7,537,889, titled “Assay for quantitation of human DNA using Alu elements,” having Sinha, et al., listed as inventors was issued on May 26, 2009 (“the Sinha '889 patent”) and the entire content is hereby incorporated by reference. The Sinha '889 patent used paired primer inter-Alu PCR to as an assay for determining presence of human DNA in a sample in which non-human DNA may also be present and for quantitating such human DNA. The assays were based on detection of multiple-copy Alu elements recently integrated into the human genome that are largely absent from non-human primates and other mammals. The Sinha '889 patent also used only a paired primer PCR system, which yielded only limited banded patters of PCR products.

The inventions described in the Sinnett'649 patent and the Sinha '889 patent have utilized traditional PCR and Alu-elements to yield paired primer PCR products. In contrast, the invention described herein combines inter-Alu PCR amplification with one to several Alu consensus seuquence-based PCR primers, preferably comprising both Head-type primer where chain elongation during PCR proceeds through, and in the direction of, the 3′-Head of the Alu element, and Tail-type primer where chain elongation during PCR proceeds through, and in the direction of, the 5′-Tail of the Alu element combined with massively parallel DNA sequencing for analyzing a huge number of regions of the genome for genetic variations. More specifically, a multitudinous amplification of different inter-Alu sequences is used to provide an unexpectedly large extended array of unique inter-Alu amplicons that encompass a huge number of segments of the human genome. This application describes for the first time, a multi-primer generated array of inter-Alu gene-enriched amplicons that has been coupled with massively parallel next generation sequencing to give an unexpectedly large array of inter-Alu sequences for a comprehensive analysis of sequences and structure variations in segments containing 10 Mb in more than 8,000 different genes for genic features associated with a trait.

Previous studies have shown that AluY subfamily insertions result in genome instability that may contribute to a variety of genetic diseases. Thus the vicinities of AluY element insertions in the human genome constitute potential recombination hotspots of possible importance to disease etiologies. Moreover, Alu elements are estimated to harbor up to 33% of the total number of CpG sites in the human genome, and the level of CpG site methylation is reported to be significantly decreased especially in AluY subfamily sequences. It follows that coupling inter-Alu PCR using Alu, and especially AluY, consensus sequence-based PCR primers to next generation sequencing can capture simultaneously a wide range of Alu- and AluY-vicinal DNA sequences for the efficient detection of SNPs, point mutations, sequence indels and DNA CpG loci methylation of potential significance to disease etiologies. By using the amplification power of PCR, the method requires only very small amounts of sample DNA. By taking advantage of the widespread distribution of Alu elements in the genome, especially in genic regions, the method most economically requires only a small number of Alu consensus sequence-based PCR primers. These important advantages in conjunction enable the generation of quality sequence data from high-throughput sequencing at low cost and low DNA sample requirement for thousands of genomic segments, enriched in genic sequences, throughout all the chromosomes of the human genome.

### SUMMARY

A first aspect of the invention is an array of inter-Alu gene-enriched amplicons produced by a polymerase chain reaction (“PCR”) process. The process comprises combining an inter-Alu-PCR-mixture containing: any number of Head-type Alu consensus sequence-based primers; any number of Tail-type Alu consensus sequence-based primers; a genomic DNA template isolated from cells; and a PCR-extension mix. The PCR extension mix comprises: a set of free deoxynucleotide triphosphate A,G,T and C bases; a thermostable DNA polymerase; and a buffer solution, whereby, the combination of primers, DNA template; and PCR extension mix comprises an inter-Alu-PCR-mixture. After making the inter-Alu-PCR mixture, an inter-Alu PCR cycle program is used in connection with a PCR machine for a period of time to produce the array of inter-Alu gene-enriched amplicons. One preferred embodiment of the invention uses a genomic DNA template isolated from blood cells. Another preferred embodiment uses a process step of verifying the production of a huge array of inter-Alu genic dense amplicons by visualizing them as a non-banded smear pattern on a gel electrophoretogram. A third preferred embodiment utilizes different single primer alone, SEQ ID No.: 2 (5′ TGGTCTCGAT CTCCTGACCT C 3′) (AluY66H21) as the Head-type AluY consensus sequence-based primer, SEQ ID No.: 1 (5′GAGCGAGACT CCGTCTCA 3′) (AluY278T18) as the first Tail-type AluY consensus sequence-based primer, SEQ ID NO.: 3 (5′ AGGCTGAGGC AGGAGAATG 3′) (AluT-T) as the second Tail-type AluY consensus sequence-based primer, SEQ ID No.: 7 (5′ GGCTCAAGCG ATCCTC 3′) (AluJo56H16) as the Head-type AluJo consensus sequence-based primer, SEQ ID No.: 8 (5′ TATGATCGTG CCACTG 3′) (AluJo232T16) as the Tail-type AluJo consensus sequence-based primer, SEQ ID No.: 9 (5′ ACCTCAGGTG ATCCAC 3′) (AluSq56H16) as the Head-type AluSq consensus sequence-based primer, SEQ ID No.: 10 (5′ AACAAGAGCG AAACTC 3′) (AluSq263T16) as the Tail-type AluSq consensus sequence-based primer, and SEQ ID No.: 6 (5′ AGCGAGACTC CG 3′) (R12A/267) as the general Alu consensus sequence-based primer. A fourth preferred embodiment generally utilizes two or more different Head-type primers. A fifth preferred embodiment generally utilizes two or more different Tail-type primers. A sixth preferred embodiment generally utilizes any combination of two or more primers that include at least one Head-type and one Tail-type primer.

A second aspect of the invention a method of identifying relevant features of inter-Alu sequences that are genetically associated with a trait. The method comprises: (a) selecting a paired of discovery genomic samples, wherein the trait is expressed in the first genomic sample but not in the second genomic sample; (b) generating an array of inter-Alu amplicons for each of the paired discovery samples; (c) sequencing the array of inter-Alu amplicons using a next generation sequencing technique forming next generation massively parallel sequencing; (d) storing the next generation sequencing results each of the paired discovery genomic samples in a computer readable format; (e) identifying relevant inter-Alu DNA sequences where an identifiable sequence feature is present in only one of the paired genomic samples. As more and more paired genomic samples are analyzed, the observation of a strongly positive correlation between a given trait and a particular sequence feature will furnish evidence that the particular sequence feature is associated with the trait. On this basis, the particular sequence feature will provide a diagnostically useful indicator that any genome displaying the particular sequence feature would be prone to express the given trait.

In a first preferred embodiment of this method, the particular sequence feature comprises a single nucleotide polymorphism or point mutation. In a second preferred embodiment, the particular feature comprises an insertion or deletion (viz. indel) of one or more bases in the DNA. In a third preferred embodiment, the particular feature comprises a chromosomal rearrangement. In a fourth preferred embodiment, the particular feature comprises a loss of heterozygosity. In a fifth preferred embodiment, the particular feature comprises a copy number variation (CNV). In a sixth preferred embodiment, the particular feature comprises the gain or loss of a chromosome or an arm or a segment of a chromosome. In a seventh preferred embodiment, the particular feature comprises an altered level of localized C-G doublet methylation. In an eigth preferred embodiment, the particular feature comprises an altered level of dispersed C-G doublet methylation. In a ninth preferred embodiment, the particular feature comprises an alteration in the amino acid sequence of a protein. In a tenth preferred embodiment, the particular feature comprises an alteration in the post-translational modification of a protein. In an eleventh preferred embodiment, the particular feature comprises a frameshift in the amino acid sequence of a protein. In a twelfth preferred embodiment, the particular feature comprises an alteration in the base sequence of an RNA. In a thirteenth preferred embodiment, the particular feature comprises an alteration in the post-transcriptional modification of an RNA. In a fourteenth preferred embodiment, the given trait comprises a disease, a disease outcome, or a disease susceptibility. In a fifteenth preferred embodiment, the trait comprises the response to a drug or pharmaceutical in terms of the efficacy or side effects or both elicited by the drug. In a sixteenth preferred embodiment, the trait comprises a personality characteristic. A seventeenth preferred embodiment comprises selecting the features of one or more genetic elements to be derived from DNA sequence data, genetic linkage data, gene expression data, antisense RNA data, microRNA data, proteomic data, or a combination thereof.

### BRIEF DESCRIPTION OF DRAWINGS

Below are the descriptions of drawings and embodiments of the present invention.

FIG. 1 shows an illustration of a single inter-Alu amplicon that is produced using any general Alu or Alu subfamily consensus sequence PCR primer set and isolated genomic DNA. The inter-Alu amplicon contains a genic region containing an SNP site flanked by non-genic regions.

FIG. 2 shows an illustration of an AluY element with a 5′ (head)-half (white) and a 3′ (tail)-half (black), and a poly-A-tail (“An”). The annealing positions of two AluY consensus primers are shown as AluH-H and AluT-T with arrows that respectively indicate the head and tail portions of the AluY element and the directions of their extension in a PCR cycle.

FIG. 3 shows an illustration of the nucleotide sequences of two AluY consensus primers. The “tail-to-tail” or “tail-type” amplification primer AluT-T has a 19-base sequence of 5′-AGGCTGAGGCAGGAGAATG-3′ (SEQ ID 3) corresponding to base positions 182 to 200 on AluY; while the “head-to-head” or “head-type” amplification primer (AluH-H) has a 21-base sequence of 5′-TGGTCTCGATCTCCTGACCTC-3′ (SEQ ID 2, or AluY66H21) corresponding to base positions 66 to 86 on the AluY. During PCR, the AluT-T primer will be extended by thermostable DNA polymerase in the direction of, and proceeding beyond, the 3′ tail of the AluY element, whereas the AluH-H primer will be extended in the direction of, and proceeding beyond, the 5′ head of the AluY element, as indicated by their respective arrows. Due to the uneven distribution of AluY elements in the genome, each having a 5′ head and a 3′ tail, segments with varying inter-Alu distances between two adjacent Alu's will be amplified by inter-Alu PCR.

FIG. 4 shows an illustration representation of a gel electrophoretogram of amplicons obtained from inter-Alu PCR. Left: banded gel pattern obtained using only the AluT-T primer by itself; Middle: 1 kb DNA markers (from Fermentas, a subsidiary of Thermo Scientific); Right: banded gel pattern obtained using only the AluH-H primer alone. Both primers gave rise to amplicons ranging from 250 bp to 2 kb in size in inter-Alu PCR. Arrows represent the separate the fragment ranges excised for sequencing.

FIG. 5 shows an illustration of a sequence and location of AluYT1 primer on the AluY element. AluYT1 primer has an 18-base sequence of 5′-GAGCGAGACTCCGTCTCA-3′ (SEQ ID 1) corresponding to base positions 278 to 295 of the AluY consensus sequence. This primer was employed for the detection of cancer mutations.

FIG. 6 shows an illustration of the inter-Alu PCR schemes using three different primers singly or in combination. Part 1—shows how the use of a head-type AluH-H alone would arrange on a DNA template allowing amplification of the intervening sequence of the Alu elements (“inter-Alu”) between two adjacent Alu 5′ heads, wherein the arrows show amplification direction. Part 2—shows how the use of tail-type AluYT1 or the tail-type general Alu consensus primer R12A/267 (5′-AGCGAGACTCCG-3′) (SEQ ID 6) alone can amplify the inter-Alu sequences between two adjacent Alu 3′ tails, wherein the wherein the arrows show amplification direction. When PCR is conducted in the presence of all three of these primers, the amplification of the inter-Alu sequences shown in Part 1 and Part 2 occur in addition to head-to-tail (Part 3) and tail-to-head (Part 4) types of amplification. As such, the variety of amplicons obtained with the three primer amplification is the sum of amplifications found in the entire FIG. 6 and can be visualized as a non-banded smear on an electrophooretogram.

FIG. 7 shows an illustration of a gel electrophoretograms of amplicons obtained from individual patients having cancer and using the tail-type AluYT1 primer. The electrophoretogram is shown as having a banded pattern. Each pair of lanes (F, L, G or W) compares the inter-Alu PCR products amplified from paired cancer and control DNA's extracted from respectively glioma tissue and peripheral blood (containing normal white blood cells) of the same patient. F: patient with primary glioblastoma; L: another patient with primary glioblastoma; G: patient with secondary glioblastoma; W: patient with anaplastic oligodendroglioma. The right hand lane is 1 kb DNA markers (from Fermentas). Arrows point to representations of visible band difference between glioma and control DNA.

FIG. 8A illustrates a flow diagram (800) for the isolation of genomic DNA (810), preparing three primer-PCR mixture (820), generating gene-enriched inter-Alu sequences (830) and visualizing an array of genic dense amplicons (840) obtained from inter-Alu PCR performed in the presence of all three of AluH-H, AluYT1 and R12A/267, yielding a smeared gel pattern rather than a banded gel pattern when visualized using UV light. (Note: visualization is Although not wanting to be bound by theory, the smeared gel pattern appears due to the vastly increased variety of amplicons when compared to the Head-type only, or Tail-type only amplicons, which give banded patterns.

FIG. 8B illustrates a flow diagram (801) for the identification of cells having a trait (845), and/or cells not having a trait (846); followed by the isolation of genomic DNA (850) from these samples; the preparation of a three primer PCR (855); the generating of genic dense inter-Alu sequences using PCR (860); the sequencing of the generated gene-enriched inter-Alu sequences using massively parallel sequencing techniques (865); the saving of sequence information in a computer readable form; and the comparing of genic dense amplicons sequences from cells having a trait and cells not having a trait and/or a sequence database (880).

FIG. 8C shows an illustration of a gel electrophoretogram of amplicons obtained from inter-Alu PCR performed in the presence of all three of AluH-H, AluYT1 and R12A/267, yielding a smeared gel pattern rather than a banded gel pattern on account of a vastly increased variety of amplicons. In this example, the left lane shows a non-banded smear comprising the inter-Alu genic amplicon from DNA isolated from an anaplastic oligodendroglioma; and the middle lane shows a non-banded smear comprising the inter-Alu genic amplicon from normal peripheral blood. Both samples were isolated from the same patient. The right lane illustrates a banded 1 kb DNA marker (Fermentas).

FIG. 9 shows an illustration of the positions and directions of amplification for two AluY consensus sequence-based PCR primers CH11 and CT11, which are both 11 bp long, and based on positions 113-123 and 160-170 respectively of the AluY consensus sequence. CH11 is a head-type primer and amplifies towards 5′ direction, whereas CT11 is a tail-type primer and amplifies towards 3′ direction.

FIG. 10 shows an illustration of the 113-123 bp and 160-170 bp segments of AluY consensus sequence, and the CT11 and CH11 primers. The sequence of CT11 is complementary to the complement segment of 160-170 bp of AluY after the two “C” residues in the complement segment have been replaced by “U”, in keeping with the conversion of all “C” on genomic DNA outside of CpG di-deoxynucleotides to “U” upon bisulfite treatment. Likewise, the sequence of CH11 is complementary to 113-123 bp of AluY after the three “C” residues in 113-123 bp have been replaced by “U”. In both the CT11 and CH11 sequences, all the “A” residues that result in response to the “C” to “U” conversion on genomic DNA are enclosed inside square boxes.

FIG. 11 shows an illustration of the primers CH11 and CT11 capable of generating amplicons. More specifically, Part 1 shows amplification using CH11 alone is capable of generating head-to-head sequences between two AluY 5′ heads. Similarly, Part 2 shows the amplification using CT11 alone capable of generating tail-to-tail amplifications between two AluY 3′ tails. When both CH11 and CT11 are added to the same inter-Alu PCR reaction, head-to-tail and tail-to-head amplifications are also obtained as shown in Part 3 and Part 4. The variety of amplicons obtained with the modified CH11 and CT11 primers are useful for identifying DNA methylation differences in gene-enriched amplicons.

FIG. 12A shows an illustration of the inter-Alu PCR sequencing outcome of bisulfite treated genomic DNA. Although not wanting to be bound by theory, only about 3 μg of the inter-Alu PCR amplicons are required for high-throughput next-generation sequencing to detect C-methylations on the bisulfite treated genomic DNA: Part 1 shows two sequences that are only different by a C-methylated bases. Part 2 shows DNA treated with bisulfite converts all G nucleotide bases to U nucleotide bases and methylated C bases are not affected. Part 3 shows that all originally methylated “C” residues on the bisulfite treated DNA give rise to “C” residues on the amplicon sequences, whereas all originally unmethylated “C” residues on the bisulfite treated DNA give rise to “T” residues on the amplicon sequences. These divergent outcomes arising from methylated and unmethylated “C” is highlighted by the bold-font “C” on the bottom line on the left hand side of the figure, versus the bold-font “T” on the right hand side.

FIG. 12B illustrates a flow diagram (1200) for the identification of methylated C bases in cells having a trait or not having a trait (1210 and 1220); followed by the isolation of genomic DNA (1230) and treatment of the isolated DNA with sodium bisulfite (1235). The preparation of a PCR mixture having primers with selected c or G-nucleotides replaced with A-nucleotides (1240); generating of gene-enriched inter-Alu sequences using PCR (1250); the sequencing of the generated gene-enriched inter-Alu sequences using massively parallel sequencing techniques (1260); the saving of sequence information in a computer readable form (1270); and the comparing of gene-enriched amplicons sequences from normal, abnormal and/or a sequence database (1280).

FIG. 13 shows the Inter-Alu sequences in the human genome. Panel A—shows the length distribution of inter-Alu distances between two adjacent Alu-transposons in the reference human genome. Heights of bars represent number of adjacent Alu pairs in the human genome at different inter-Alu distances. Subtraction of empty column at <200 bp representing short inter-Alu sequences that were removed from analysis during library construction for next generation sequencing leaves the solid columns of inter-Alu sequences of varying lengths capturable from the genome. The total sum of the solid columns up to an inter-Alu distance of 6 kb, 8 kb or 10 kb equals 1.10 Gb, 1.36 Gb or 1.58 Gb, respectively. Panel B—shows Inter-Alu sequences of 0.2-6.0 kb in length that are in principle capturable by the three Alu-consensual primers AluY278T18, AluY66H21 and R12A/267. Such capturable amplicons amount to ˜14 Mb if no mismatch is allowed between the consensual primers and template sequences, or ˜106 Mb if one mismatch is allowed per primer. The graph shows the length distribution of the latter 106 Mb.

FIG. 14 shows the amplicon range of inter-Alu PCR. The different lanes show gel electrophoretograms of amplicons obtained using different single primer or multiple primer sets: (A) AluJo56H16 alone; (B) AluJo232T16 alone; (C) AluSq56H16 alone; (D) AluSq263T16 alone; (E) AluJo56H16 and AluJo232T16; (F) AluJo56H16 and AluSq56H16; (G) AluJo56H16 and AluSq263T16; (H) AluJo232T16 and AluSq56H16; (I) AluJo232T16 and AluSq263T16; (J) AluSq56H16 and AluSq263T16; (K) AluJo56H16, AluSq56H16 and H-type L12A/8; (L) AluJo232T16, AluSq263T16 and T-type R12A/267, (N) AluY278T18, (P) AluY278T18 and AluY66H21, (Q)-(S) AluY278T18, AluY66H21 and R12A/267, (T) AluJo56H16, AluSq56H16 and AluSq263T16, (U) AluJo56H16, AluSq263T16 and AluJo232T16, (V) AluJo56H16. AluSq263T16 and L12A/8, and (M) M.W. markers. Notably, lanes E, G, H, J, P-V, where the inter-Alu PCR was performed using both H-type and T-type primers, gave rise to a largely smeared gel. Comparison of lanes N and P showed the conversion of a banded pattern obtained using a single T-type primer to a smeared one through the addition of an H-type primer. The much stronger staining intensity of lane S relative to lane R showed that the 0.30 μM concentrations of the same three primers in S compared to 0.10 μM in R resulted in increased amounts of amplicons. The primers AluJo56H16 (5′-GGCTCAAGCGATCCTC-3′) (SEQ ID 7), AluJo232T16 (5′-TATGATCGTGCCACTG-3′) (SEQ ID 8), AluSq56H16 (5′ACCTCAGGTGATCCAC-3′) (SEQ ID 9), and AluSq263T16 (5′-AACAAGAGCGAAACTC-3′) (SEQ ID 10) were based on AluJo and AluSq consensus sequences. H-type L12A/8 was an Alu consensus primer suggested earlier for inter-Alu PCR. AluY278T18, AluY66H21 and T-type R12A/67 are described in Methods.

FIG. 15 shows a correlation between number of Alu elements and sequences captured. Relationship between the number of Alu elements occurring on individual human chromosomes (blue bars) and the amount of AluScan-captured sequences of white blood cell DNA mapped to the different chromosomes (red triangles). Correlation coefficient was 0.939 between these quantities.

FIG. 16 shows Density distributions of captured sequences mapped on chromosomes. The lengths of genomic sequences (are shown above each chromosome as vertical spikes) mapped within 10-kb non-overlapping windows were plotted along chromosomes 1-22, X and Y using Human Genome Graphs (http://genome.ucsc.edu/cgi-bin/hgGenome) for (A) white blood cell DNA and (B) glioma DNA.

FIG. 17A shows Genetic variations identified in the white blood cell genome relative to the reference human genome. From the outside, Tracks 1 and 2: numbering and cytobands of different chromosomes. Track 3: red dots showing number of SNVs per 1 Mb window (scale: 0-60, 1 div=12). Track 4: blue dots showing number of indels of ≦30 bp per 1 Mb window (scale: 0-12, 1 div=2.4). Inner circle: green bars showing positions of structural variations in the form of two large indels at chromosomes 3 and 19. The figure was drawn using the Circos program. Must be changed?

FIG. 17B shows somatic mutations in glioma cells. From the outsider, Tracks 1 and 2: numbering and cytobands of different chromosomes. Track 3: black bars showing the LOH-enriched loci in chromosomal regions, most prominently in 1p, 9p, 9q and 19q. Track 4: blue bars showing number of somatic indels of ≦30 bp per 5-Mb window (scale: 0-6). Track 5: red bars showing number of somatic SNVs per 5-Mb window (scale: 0-8). The cytogenetic locations of seven potential SNV hotspots displaying a density of somatic SNVs>4, viz. greater than the sum of the Mean (=1.5) plus two-times the standard deviation (=1.9), per 5-Mb window are indicated as 11q13.4, 12q13, 16p13.1 etc. Must be changed?

FIG. 18 shows a table of AluScan sequence outputs from control and glioma DNA.

FIG. 19 shows the distributions of SNVs and indels among different genomic regions. Header: Control SNV=SNVs in control DNA relative to reference human genome GRCh37. Glioma SNV=SNVs in glioma DNA relative to reference human genome. Somatic SNV=SNVs between control and glioma DNAs. Control Indel=indels in control DNA relative to reference human genome. Glioma Indel=indels in glioma DNA relative to reference human genome. Somatic Indel=indels between control and glioma DNAs. LOH SNV=LOHs between control and glioma DNAs.

FIG. 20 shows the distribution of SNVs among different classes of nucleotidyl changes. X-axis shows six different classes of possible nucleotidyl change in SNV, and Y-axis shows percentage of each class. Columns represent SNVs in control DNA relative to reference human genome (tripe), in glioma DNA relative to reference human genome (white), and between the paired control and glioma DNAs (black).

FIG. 21 shows the distribution of LOHs on different chromosomes. LOH regions are indicated in red, Non-LOH regions in yellow and unmapped regions in black, on horizon line in the diagram for each chromosome. Grey dots represent frequencies of non-reference alleles, found in either control or glioma SNVs that were not represented in the reference human genome. X axis shows position along each chromosome, and Y axis the non-reference allele frequency.

FIG. 22 shows the genic regions in potential SNV hotspots revealed by AluScans. Header: Potential Hotspot=chromosomal location of each potential SNV hotspot shown on FIG. 17B. SNV position=positions of different genic SNVs in an indicated hotspot. Region=location of a genic SNV in a particular gene shown in ‘Gene’ column. Gene=name of gene in an indicated hotspot containing an SNV.

### DESCRIPTION OF PREFERRED EMBODIMENTS

Before describing one aspect of the present invention in detail, it is to be understood that this invention is not limited to particular compositions or methods for making compositions, which may vary. It is also to be understood that the terminology used herein is for the purpose of describing particular embodiments only, and is not intended to be limiting. In addition, before describing detailed embodiments of the invention, it will be useful to set forth definitions that are used in describing the invention. The definitions set forth apply only to the terms as they are used in this patent and may not be applicable to the same terms as used elsewhere, for example in scientific literature or other patents or applications including other applications by these inventors or assigned to common owners. Additionally, when examples are given, they are intended to be exemplary only and not to be restrictive.

It must be noted that, as used in this specification and the appended claims, the singular forms “a,” “an” and “the” include plural referents unless the context clearly dictates otherwise. Thus, for example, reference to “a pharmacologically active agent” may also include a mixture of two or more such compounds, reference to “a base” may also include mixtures of two or more bases, and the like.

In describing and claiming the present invention, the following terminology will be used in accordance with the definitions set out below.

The term “Alu-element” as used herein encompasses a short stretch of DNA originally characterized by the action of the Alu (Arthrobacter luteus) restriction endonuclease. Alu elements of different kinds occur in large numbers in primate genomes. In fact, Alu elements are the most abundant Transposable elements in the human genome. They are derived from the small cytoplasmic 7SL RNA, a component of the signal recognition particle. The event, when a copy of the 7SL RNA became a precursor of the Alu elements, took place in the genome of an ancestor of Supraprimates. Alu insertions have been implicated in several inherited human diseases and in various forms of cancer. The study of Alu elements has also been important in elucidating human population genetics and the evolution of primates, including the evolution of humans.

The term “genic regions” as used herein refers to regions in the genome located within a gene (genetic element) as the molecular unit of heredity. It represents specific DNA sequence carrying genetic information that has a function in the human organism.

The term “purified PCR products” as used herein refers to PCR products generated from inter-Alu PCR and treated with ethanol or other purification kits to remove any excess primers, enzymes, mineral oil, glycerol and salts.

The term “inter-Alu regions” as used herein refers to the DNA sequences, positioned between two Alu elements, that are amplified during inter-Alu PCR. Since Alu elements are widespread in the human genome, inter-Alu regions that come to be PCR amplified in the presence of multiple Alu-consensus sequence-based primers could cover a substantial portion of the entire genome.

The term “quality” as used herein refers to two attributes of the inter-Alu PCR amplicons: the amount of amplicons produced, and the usefulness of their sequence data e.g. the proportion of genic sequences among the PCR products, the average coverage provided by these products over different regions of the genome etc.

The term “nanogram level of genomic DNA” as used herein refers to the submicrogram amounts of DNA needed for inter-Alu PCR followed by next generation sequencing.

The term “Alu consensus sequence-based primer” as herein refers to the inter-Alu PCR primers complementary to Alu consensus sequences, typically 10-20 bases in length.

The term “AluY consensus sequence-based primer” as herein refers to the inter-Alu PCR primers complementary to AluY subfamily consensus sequences, typically 10-20 bases in length.

The term “white bands” as used herein refers to amplicons with discrete ranges of length obtained from inter-Alu PCR, which upon agarose gel electrophoresis, ethidium bromide staining and UV visualization give rise to white banded patterns.

The term “thermo-stable DNA polymerase” as used herein refers to the DNA polymerase used in inter-Alu PCR, which can be Taq polymerase, KOD polymerase or other polymerases used in DNA amplification.

The term “direction of amplification” as used herein refers to the direction of PCR amplification proceeding forward through either the 5′ (head) or 3′ (tail) end of an Alu element annealed to by an Alu consensus sequence-based primer.

The term “Massive Parallel Sequencing” may also refer to an advanced fluorescent-labeled sequencing technology capable of producing giga-bases of sequence information in a single run.

The term “tail-to-tail” as used herein refers to the amplification of the inter-Alu segment between one Alu 3′ end and an adjacent Alu 3′ end.

The term “head-to-head” as used herein refers to the amplification of the inter-Alu segment between one Alu 5′ end and an adjacent Alu 5′ end.

The term “head-to-tail” as used herein refers to the amplification of the inter-Alu segment between one Alu 5′ end and an adjacent Alu 3′ end.

The term “tail-to-head” as used herein refers to the amplification of the inter-Alu segment between one Alu 3′ end and an adjacent Alu 5′ end.

The term “exon capture” as used herein refers to the capture of exons using hybridization and other methodologies for sequencing.

The term “CpG loci” as used herein refers to sites on DNA with a 5′-CpG-3′ sequence. In mammals, 70% to 80% of CpG cytosines are methylated.

The term “Massive Parallel Sequencing” as used herein encompasses several high-throughput approaches to DNA sequencing; it is also called “next-generation sequencing” (“NGS”) or “second-generation sequencing.” Some of these technologies emerged in late 1996 and became commercially available since 2005. These technologies use miniaturized and parallelized platforms for sequencing of 1-100 million of short reads (50-400 bases). Many NGS platforms differ in engineering configurations and sequencing chemistry. However, they share the technical paradigm of massive parallel sequencing via spatially separated, clonally amplified DNA templates or single DNA molecules in a flow cell. This design is very different from that of Sanger sequencing, which is also known as capillary sequencing or first-generation sequencing that is based on electrophoretic separation of chain-termination products produced in individual sequencing reactions. The term “Massive Parallel Sequencing” may also refer to an advanced fluorescent-labeled sequencing technology capable of producing giga-bases of sequence information in a single run.

The term “Amplicon” as used herein refers to a piece of DNA formed as the product of natural or artificial amplification events. For example, it can be formed via polymerase chain reactions (“PCR”) or ligase chain reactions (“LCR”), as well as by natural gene duplication. Traditionally, the artificial amplification of a locus of Alu-element insertion could be selected, amplified and evaluated in terms of size of the fragment, which are usually visualized as a banded pattern in electrophoretic separation techniques. In the case of this invention, a three primer PCR amplification would usually suffice to lead to a wide enough array of inter-Alu-amplicons of varying sizes that, upon size separation on gel electrophoresis, will give rise to a non-banded smear. In turn, whenever any set of PCR primers produces a non-banded smear, there will be assurance that a wide sprctrum of amplicons of varying sizes has been generated.

The term “GeneRanker” as used herein refers to a program allowing characterization of large sets of genes by making use of annotation data from various sources, like Gene Ontology or Genomatix proprietary annotation. Overrepresentation of different biological terms within the input are calculated and listed in the output together with the respective p-value. The algorithm behind GeneRanker is based on the paper of Gabriel F. Berriz et. al. (2003), Characterizing gene sets with FuncAssociate, Bioinformatics 19, 2502-2504 (PubMed: 14668247), the entirety of these papers are hereby incorporated by reference.

The term “The Sequence Read Archive” or “Short Read Archive” as used herein refers a bioinformatics database and a collaboration between the European Bioinformatics Institute, the National Center for Biotechnology Information, and the DNA Data Bank of Japan. It provides a public repository for the “short reads” generated by High-throughput sequencing. The reads from high-throughput or ‘next-gen’ sequencers are typically less than 1,000 bp of DNA sequence.

The term “Burrows-Wheeler Aligner” (“BWA”) as used herein refers to an efficient program that aligns relatively short nucleotide sequences against a long reference sequence such as the human genome. It implements two algorithms, bwa-short and BWA-SW. The former works for query sequences shorter than 200 bp and the latter for longer sequences up to around 100 kbp. Both algorithms do gapped alignment. They are usually more accurate and faster on queries with low error rates. Please see the BWA manual page for more information.

The term “NimbleGen Sequence Capture Products” as used herein allows parallel enrichment of many genomic target regions in a single experiment for sequencing with the GS FLX and GS Junior Systems. These types of systems allow a user to target any region of interest and capture up to 50 Mb custom regions or the whole human exome with high coverage and specificity. Data can be generated and empirically optimized using a validated capture design algorithm. Important Variants can then be detected. The unique long reads from the GS FLX and GS Junior Systems enable easy detection of multi-base insertions and deletions, complex structural variations, improve coverage in repetitive regions, and provide haplotype information. The data can then be easily analyzed using a dedicated GS Reference Mapper software report having variant locations, amino acid changes in coding regions, and known SNP information. In addition, the software can generates capture performance metrics, such as percentage reads in target regions.

The term “SeqCap EZ Library” as used herein refers to a commercially available solution-based capture method that enables enrichment of the whole exome or custom target regions of interest in a single reaction. SeqCap EZ Choice Libraries enable enrichment of custom regions of interest and are offered in two configurations capable of capturing up to 7 Mb of target regions with a single design and 50 Mb of target regions with a single design. Other similar systems can also be utilized without departing from the spirit and scope of this invention.

One aspect of the present invention is directed to the detection of sequence and structural features in genomic regions, enriched in genic regions. The method employs inter-Alu PCR using only a single or a small number of AluY or other Alu consensus sequence-based PCR primers to capture a myriad of genomic sequences, positioned between two Alu elements and enriched in genic sequences, for massively-parallel sequencing. The method is highly economic in terms of the nanogram (“ng”) range of sample DNA required, and the generation of a huge range of high-quality amplicons totaling more than 10 megabases of DNA sequence. These amplicons are enriched in genic regions, and can be methodically varied through the employment of different sets of AluY and other Alu consensus sequence-based PCR primers.

Another aspect of the present invention involves the detection of single nucleotide polymorphisms (“SNP”), point mutations, insertion/deletions (indel) and CpG loci DNA methylation. The method uses inter-Alu PCR in conjunction with massively-parallel sequencing technology for the detection of sequence and structural variations in the genome. Because Alu elements are distributed in primate genomes and tend to accumulate in gene-rich regions, inter-Alu PCR can provide an effective pre-sequencing capture of inter-Alu sequences enriched with genic sequences across the genome. The quality of DNA amplicons obtained from inter-Alu PCR is six times better than direct use of genomic DNA templates in terms of yield of DNA sequences and coverage of genic regions in the genome This method enables the use of only submicrogram levels of genomic DNA samples for the purpose of massively-parallel sequencing directed to the detection or discovery of genetic variations (SNPs, point mutations, indels and CpG loci DNA methylation). One embodiment of the present invention exploits the impact of AluY element insertions in causing genomic instabilities and recombination hotspots, where the frequencies of SNPs including possibly disease-associated SNPs are enhanced By employing inter-Alu PCR with AluY consensus sequence-based primers, DNA sequences in the inter-Alu regions are selectively amplified Cycles of PCR are performed with thermo-stable DNA polymerase, and DNA replication would be carried out with the addition of free deoxynucleoside triphosphates (A, G, C, T).

The exponential amplification ability of PCR enables the use of only submicrogram quantities of genomic DNA produce enough inter-Alu amplicons for analysis by massively-parallel sequencing. At the same time, due to the structural similarity of different Alu repeat elements and their abundance (accounting for more than 10% of the human genome), use of a single AluY specific primer can generate a range of variously sized PCR amplicons amplified from different regions in the genome, and use of multiple AluY- and other Alu-consensus primers can generate a multitude of such amplicons. Thus, when a single Alu-consensus primer is employed, agarose gel electrophoresis and ethidium bromide staining reveals the PCR amplicons obtained mainly as discrete bands upon UV visualization. When multiple primers are employed, the amplicons become so numerous that they appear as continuous smears on the gel, consisting of a myriad of inter-Alu sequences originating from all kinds of chromosomal locations in the human genome. Since a large number of Alu repeats are located in or near genic regions of the genome, massively-parallel sequencing of the amplicons show that the amplicons come to be enriched up to 40% in genic sequences, even though genic sequences comprise only 25% of the whole genome. Most of the SNPs detected among the amplicons are located in the Alu sequence or its flanking regions. The method therefore provides a useful enriching tool for the monitoring and/or discovery of known and novel genic SNPs and indels in the genome.

Another embodiment of this invention employs the above-stated method to detect genetic variations that are specific to different disease states, especially point mutations, indels and loss of heterozygosity occurring in the introns and exons in a cancer genome. There are an estimated 25,000 genes in the whole human genome. Among them, as many as 6,522 genes are known to be associated with cancer, accounting for 26% of total number of genes. When the present invention was employed with AluY consensus sequence-based primers to amplify cancer genomic DNA, 58% of the genes found within genic regions in the amplicons were cancer-associated In the procedure, two AluY specific primers together with the Alu-consensus sequence primer R12A/267 were jointly used as PCR primers. Through the action of thermostable DNA polymerase, the primers would be annealed to the complementary sequences throughout both strands of template genomic DNA forming primer-template hydrids. DNA replication was initiated by the addition of free deoxynucleoside triphosphates (A, G, C, T), yielding a continuous smear of amplicons on the agarose gel electrophoretogram upon UV visualization. The SNPs located on these amplicons amplified inter-Alu regions were then analyzed using massively-parallel sequencing to detect sequence and structural alterations that are potentially associated with cancer.

Still another embodiment of the present invention is based on the characteristics of Alu elements especially the AluY subfamily, viz. insertions of these elements are known to contribute to genomic instability and hotspot of recombination events, and enhanced SNP frequencies including disease-associated SNPs have been found in their vicinities. In view of this, primers specific to AluY and Alu consensus sequences are designed. During inter-Alu PCR, such primers will anneal to their complementary template DNA sequences forming primer-template hybrids. Thermo-stable DNA polymerase would synthesize a new DNA strand complementary to the DNA template strand with free deoxynucleotides (A, G, T, C) in the reaction mix. As the target fragments are exponentially amplified by PCR, the PCR products would be of higher quality than the template DNA. At the same time, due to the structural similarity of Alu repeats and its abundance (more than 10%) in the human genome, even a single AluY specific primer can amplify the sequences between adjacent pairs of Alu elements in many parts of the genome. After agarose gel electrophoresis and ethidium bromide staining, the amplicons appear in a banded pattern upon UV visualization. If multiple Alu and/or AluY-based primers are present during the PCR, a smeared gel would be routinely observed upon UV visualization on account of the myriad of different amplicons produced. In this invention, the probability of obtaining amplicons containing a genic sequence is found to be a high as 40%, even though genic regions only comprise 25% of the whole genome. With this combination of small number of PCR primers, requirement for only submicrogram levels of sample DNA, and enrichment of genic regions among amplicons, the present invention combining inter-Alu PCR and massively-parallel sequencing provides a most valuable tool for the monitoring and discovery of genic SNPs, indels and heterozygosities in the genome.

Yet another embodiment of this invention utilizes the methodology to detect genetic variations associated with different diseases, including point mutations and indels occurring in the introns and exons within the cancer genome. There are an estimated 25,000 genes in the whole human genome. Out of these, 6,522 genes are found to be associated with cancer, accounting for 26% of total number of genes. When AluY consensus sequence-based primers were applied to amplify cancer genomic DNA, 58% of the genes found in the amplicons were cancer-associated. The SNPs found on the amplicons therefore could be analyzed by next generation sequencing and analyzed for potential association with cancer. The amplicons also can be analyzed using an exon capture technique, as described below in Example 2.

Another embodiment of the present invention utilizes the above-mentioned method to assess CpG loci DNA methylation in genomic regions. DNA methylation primarily occurs in 5′-CpG-3′ di-deoxynucleotides, in which a methyl group is added to the 5′ position of the cytosine pyrimidine ring (5′C) to form 5′ mC. Many 5′ mC occur within CpG enriched Alu family repeats. It has been estimated that 33% of the total number of CpG sites are harbored on Alu elements in the human genome. For that reason, a primer pair based on AluY consensus sequence devoid of CpG sites and with different directions of amplification were employed for the inter-Alu PCR. Genomic DNA samples from cancer tissue and peripheral blood (as normal control cells) treated with sodium bisulfite were used as template DNA in the inter-Alu PCR. Because treatment with sodium bisulfite converts any unmethylated C-residue in a CpG doublet to a U-residue (which basepairs like a T-residue), but does not convert any methylated C-residue in a CpG doublet, sequencing of various inter-Alu amplicons will reveal which of their original CpG doublet were unmethylated and therefore converted to TpG, and which were methylated and therefore remain as CpG. Accordingly the method can be employed to determine within a genomic sample which CpG sites are methylated and, if so, what is the level of methylation.

A further embodiment of the present invention compares the levels of methylation of various CpG sites in a genomic sample from a normal tissue with the levels of methylation in a genomic sample from a diseased tissue, e.g., comparing the methylation in a cancer tissseu and the methylation in a normal tissue.

### EXAMPLES

The following examples are included to demonstrate preferred embodiments of the invention. It should be appreciated by those of skill in the art that the techniques disclose in the examples that follow represent techniques discovered by the inventor to function well in the practice of the invention, and thus can be considered to constitute preferred modes for its practice. However, those of skill in the art should, in light of the present disclosure, appreciate that many changes can be made in the specific embodiments that are disclosed and still obtain a like or similar result without departing from the spirit and scope of the invention.

### Example 1

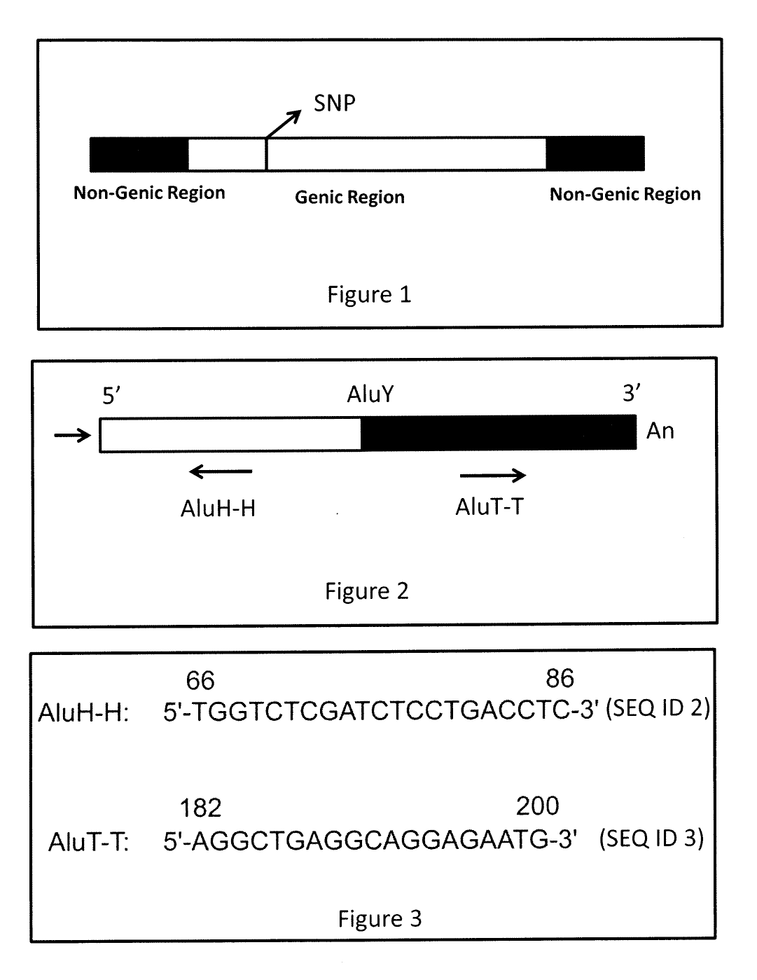

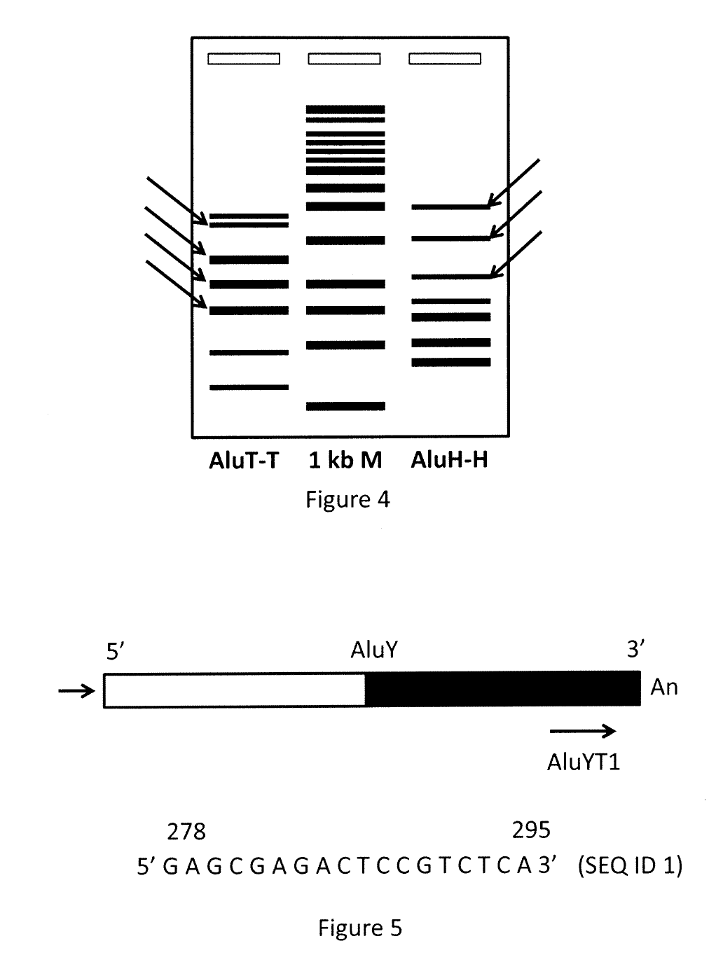

A SNP present in genic regions of the human genome, whether haploidal, homozygous diploid or heterozygous diploid, is illustrated in FIG. 1. The diagnostic identification of inter-Alu PCR is performed by amplifying genomic sequences situated in or close to Alu elements, which are enriched in genic regions. This is followed by next-generation sequencing of the amplicons to reveal the SNPs present in the genic regions among the amplicons. FIG. 2 shows the positions of two AluY consensus primers annealed to an AluY element, and their directions of amplification in PCR. Now referring to FIG. 3, which shows both the sequences of two AluY consensus primers and their corresponding base positions on AluY. During inter-Alu PCR, these AluY consensus sequence-based primers will anneal to the complementary template sequences on genomic DNA, and undergo chain elongation in the presence of free deoxynucleotide triphosphate A, G, T and C, and a thermo-stable DNA polymerase. Based on the orientation of Alu, one of the primers can amplify the sequence from one Alu 3′ end to another Alu 3′ end (tail-to-tail direction) whereas another primer can amplify the sequence from one Alu 5′ end to another Alu 5′ end (head-to-head direction). In each instance, the amplicons are observed in the banded electrophoretograms (FIG. 4) and can be analyzed by next-generation sequencing to identify the known or novel SNPs in the amplicons.

An example illustrating how the present invention can be employed to capture and identify intra-genic SNPs is given as follows. The first step is to prepare human genomic DNA using phenol/chloroform extraction, followed by ethanol purification. Purified DNA is diluted to a working concentration, usually 50 ng/μl. Useful AluY consensus sequence-based PCR primers are exemplified by AluT-T, which yields by itself “tail-to-tail” amplification, and AluH-H, which yields by itself “head-to-head” amplification. In the present example, each PCR reaction was performed in a final volume of 20 μl containing 4 μl 5× Mastermix (10×PCR buffer containing 500 mM KCl, 100 mM Tris-Cl, 15 mM MgCl₂), 50 mM MgCl₂ and 2.5 mM of each of dATP, dTTP, dCTP and dGTP, 1 μl 5 μM primer (AluT-T or AluH-H), 0.1 μl (0.5 unit) thermo-stable DNA polymerase, 2 μl 50 ng/μl human genomic DNA and 12.9 μl deionized water. PCR amplification included DNA denaturation at 95° C. for 5 min, followed by 35 cycles each of 30 s at 95° C., 30 s at 66.3° C. for AluH-H (or 66.8° C. for AluT-T) annealing, and 2 min at 72° C., plus finally another 5 min at 72° C. After completion of the PCR reaction, 10 μl PCR products were sampled to check for appearance and quality by agarose gel electrophoresis, ethidium bromide staining and UV visualization.

The gel electrophoretogram of PCR products obtained in each instance is shown in FIG. 4. A comparison of the banded pattern with 1 kb DNA markers indicated that the amplicons ranged from about 250 bp to about 2 kb in size. Seven amplicon-fractions ranging from 750 bp to 2 kb in size were excised from the two gels (as indicated by arrows in FIG. 4). The quantity of DNA in each fraction was >10 μg. A total of 372 Mb of DNA sequencing data from these seven fractions were obtained from massively-parallel sequencing. The Short Oligonucleotide Analysis Package (SOAPalinger) was employed for oligonucleotide alignment to assemble longer DNA sequence reads, which were then mapped to the reference human genome using BLAST alignment tool and UCSC database for SNP detection and discovery. Upon sequencing and bioinformatics analysis, the above-mentioned inter-Alu PCR run generated 374 DNA fragments, 153 of them of which were found to contain intra-genic sequences amounting to 47% of total sequencing output. Since genic regions only occupy 40% of the human genome, these results demonstrated that Alu elements preferentially accumulate in genic regions, and the inter-Alu sequences obtained from inter-Alu PCR were enriched in genic sequences. In addition, there are an estimated 25,000 genes in the human genome, 6,522 of which (viz. 26% of all genes) are known to be associated with cancer. In the present Embodiment, the genic regions of 128 genes were included in the sequence output. Out of these, 75 of them, or 58% of all the genes in the sequence output, were cancer-associated genes. Therefore the sequence output from the inter-Alu PCR run was enriched in cancer-associated genes relative to all known genes. By means of BLAST and UCSC database, a total of 262 SNPs (including those in non-genic regions) were identified in the sequence output, 42 of them were novel SNPs or point mutations. These results show that using the present invention, analysis of only 100 ng human DNA sample employing only two AluY-based PCR primers sufficed to provide novel and useful intra-genic SNP information.

### Example 2

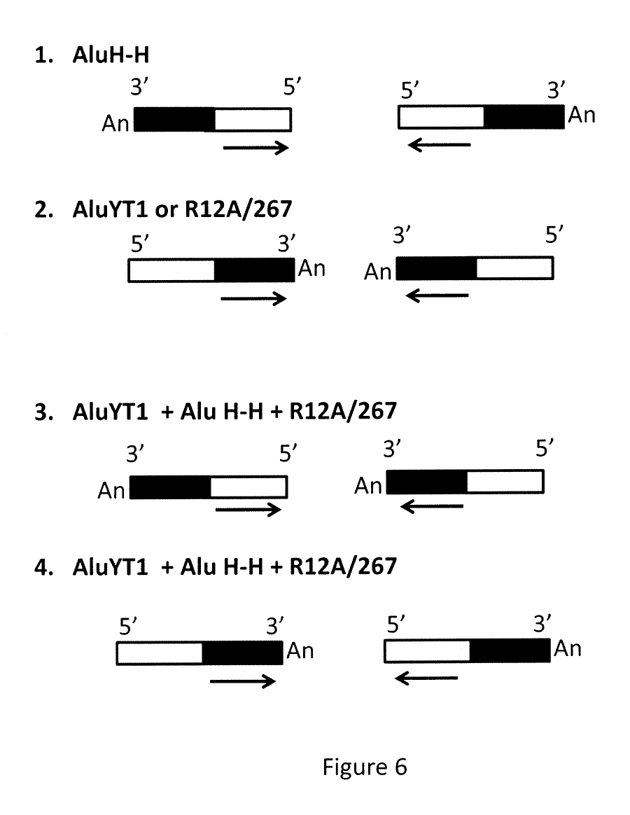

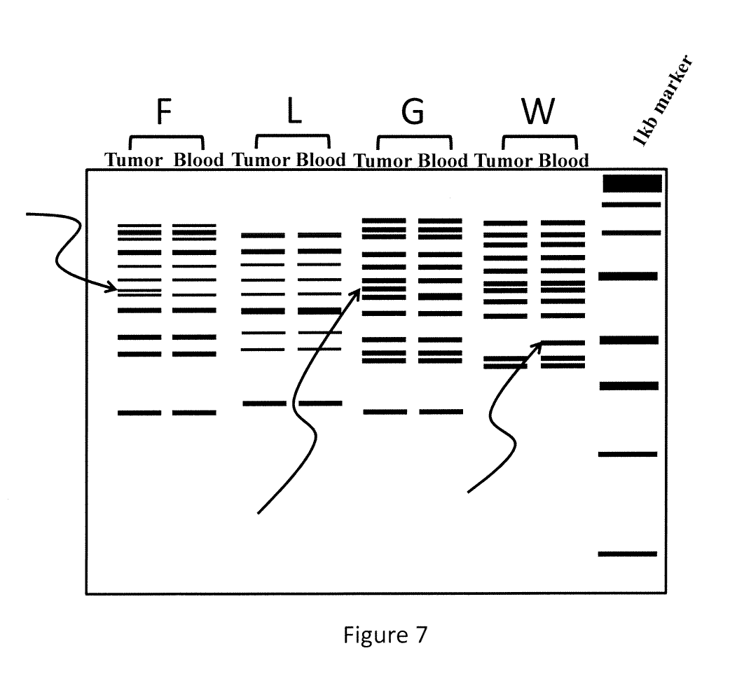

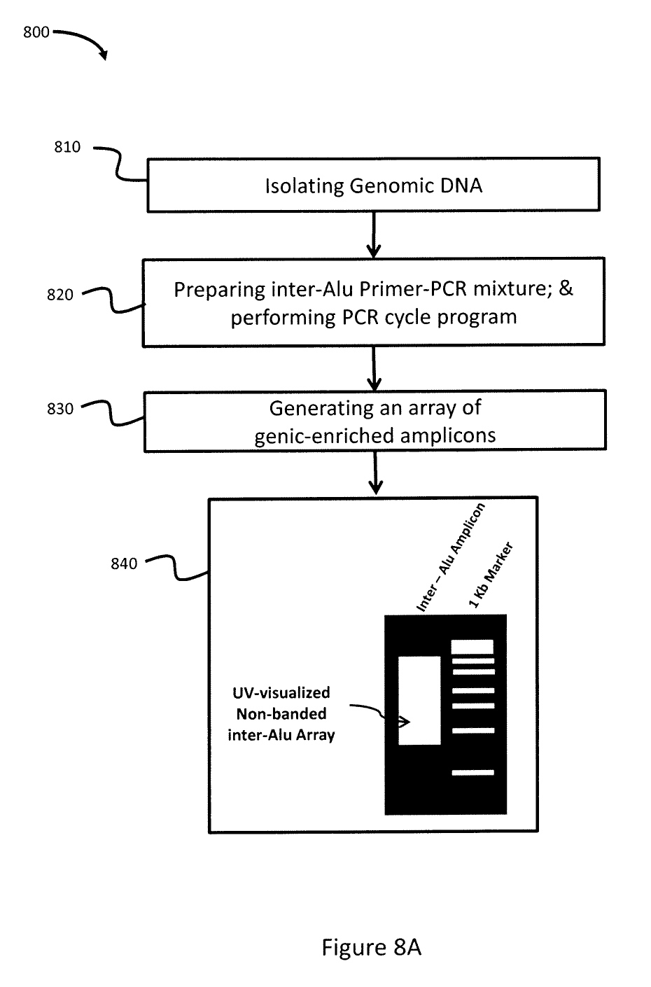

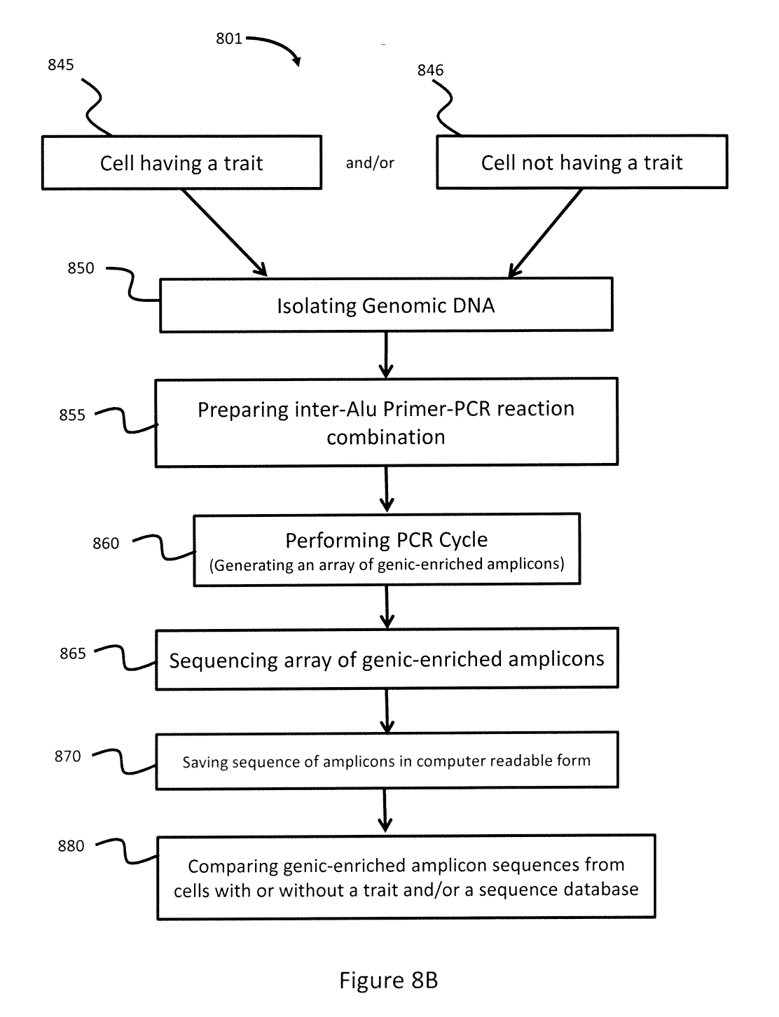

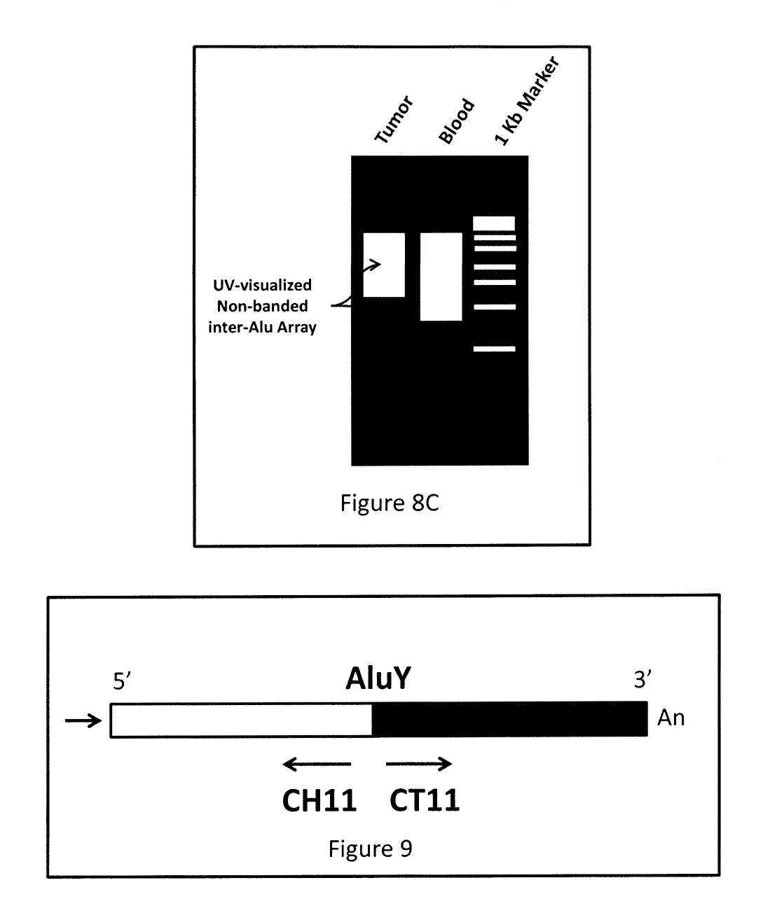

Example 2 was similar to Example 1 except that it was focused on association with multiple-gene diseases. In order to increase amplicon variety to facilitate mutation detection in cancer genome, the tail-type AluYT1 primer (viz. 5′-GAGCGAGACTCCGTCTCA-3′ (SEQ ID 1) as shown in FIG. 5) along with the aforementioned head-type AluH-H primer (5′-TGGTCTCGATCTCCTGACCTC-3′) (SEQ ID 2) and the tail-type Alu consensus primer R12A/267 (5′-AGCGAGACTCCG-3′) were employed jointly. During inter-Alu PCR, these three primers would anneal to complementary sequence sites on genomic DNA, and participate in PCR amplification. FIG. 6 shows the allowed amplification schemes of these 3 primers employed either alone or in combination. Based on the orientation of Alu, the tail-type AluYT1 or R12A/267 alone is capable of amplifying inter-Alu sequences between two Alu 3′ tails (tail-to-tail amplification), whereas the head-type AluH-H by itself is capable of amplifying inter-Alu sequences between two Alu 5′ heads (head-to-head amplification). When all these three primers are present, amplification of inter-Alu segments spanning one Alu 5′ end to an adjacent Alu 3′ end (head-to-tail) or spanning one Alu 3′ end to an adjacent Alu 5′ end (tail-to-head amplification) are obtained as well. In the present Embodiment, the AluYT1 primer was employed to amplify cancer tissue and control DNA by inter-Alu PCR, so that the size ranges of the amplicons were relatively more restricted, thus giving rise to a banded gel electrophoretogram where changes in the band pattern were more readily detected. On the other hand, AluYT1, AluH-H and R12A/267 were also employed jointly, so that the size ranges of the amplicons were greatly enhanced, giving rise to a smeared gel pattern and enabling the analysis of a vastly expanded number of amplicon sequences by next generation sequencing. These contrasting examples illustrated the flexibility of the present invention in combining inter-Alu PCR and next generation sequencing to detect altered features of the human genome in association with diseases. In the first instance employing only the AluYT1 primer, genomic DNA from cancer and control cells from the same patient was prepared by phenol/chloroform extraction, followed by ethanol purification. Purified DNA was diluted to a working concentration of 50 ng/μl. Inter-Alu PCR was performed in a final volume of 20 μl containing 4 μl 5× Mastermix (10×PCR buffer containing 500 mM KCl, 100 mM Tris-Cl, 15 mM MgCl₂), 50 mM MgCl₂ and 2.5 mM of each of dATP, dTTP, dCTP and dGTP), 1.2 μl 5 μM AluYT1 primer, 0.1 μl (0.5 unit) thermostable DNA polymerase, 2 μl 50 ng/μl human genomic DNA and 12.7 μl deionized water. PCR amplification included DNA denaturation at 95° C. for 5 min, followed by 35 cycles each of 30 s at 95° C., 30 s at 67° C., and 2 min at 72° C., plus finally another 5 min at 72° C. After completion of the PCR reaction, 20 μl PCR products were taken for electrophoresis on 1.5% agarose gel, ethidium bromide staining and UV visualization. FIG. 7 shows the gel patterns of paired amplicons from cancer tissue and peripheral blood from the same patient. Arrows indicate altered band patterns in patients F, G and W. In the second instance employing all three primers, Inter-Alu PCR was performed in a final volume of 20 μl containing 4 μl 5× Mastermix (10×PCR buffer containing 500 mM KCl, 100 mM Tris-Cl, 15 mM MgCl₂), 50 mM MgCl₂ and 2.5 mM of each of dATP, dTTP, dCTP and dGTP), 1.5 μl 5 μM, 0.9 μl 5 μM AluH-H, 0.3 μl 5 μM R12A/267, 0.1 μl (0.5 unit) thermostable DNA polymerase, 1 μl 10 ng/μl human genomic DNA and 12.2 μl deionized water. PCR amplification included DNA denaturation at 95° C. for 5 min, followed by 35 cycles each of 30s at 95° C., 30s at 57.8° C., and 2 min at 72° C., plus finally another 5 min at 72° C. After completion of the PCR reaction, 5 μl PCR product was taken for electrophoresis on 1.5% agarose gel and UV visualization. FIG. 8 shows the smeared gel electrophoretograms of amplicons from either glioma tissue and control pheripheral blood DNA. In this example, 25 ng genomic DNA generated more than 3 μg of amplicons through inter-Alu PCR. Such high yield of amplicons was favorable for massively-parallel sequencing analysis of the amplicons, producing far more genic sequences for association studies compared to using just the AluT1 primer alone. The Short Oligonucleotide Analysis Package (SOAPalinger) was employed to assemble longer DNA sequence reads that were then mapped to the reference human genome using BLAST alignment tool and UCSC database to reveal somatic mutations and indels between cancer and control DNA. Yet another application of the inter-Alu PCR amplicons described in the preceding paragraph pertains to their usage as a discovery tool in exon capture employing the adenovirus shuttle vector pETV-SD. Any gene containing introns and exons must undergo RNA splicing during transcription, which requires a splicing donor SD and a splicing acceptor SA. The procedure calls for shotgun cloning of the inter-Alu PCR amplicons into pETV-SD downstream from its exon capture sequence. Next, pooled plasmid DNA from the shotgun cloning is transfected into the retroviral packaging cell line ψ2, which provides the proteins required for propagating the vector as a retrovirus. Upon transcription of the retroviral DNA in vivo, transcripts of recombinant plasmids that contain a functional SA could undergo a splicing event with the loss of IVS. Both spliced and non-spliced viral RNAs are then packaged into virions, which after harvesting from the medium are used to infect the retroviral packaging cell line PA-317. This results in an additional round of retroviral replication and produces viral stocks of increased titer capable of infecting monkey renal COS cells, which constitutively produce the SV40 large tumor (T) antigen. The viral RNA genome is reverse transcribed and amplified as a circular DNA episome due to the presence of the SV40 origin of replication in the vector. The replicated episomal DNA is recovered from the COS cells, digested with Dpn I, and transformed into bacterial cells. Transformants are selected on agar plates containing kanamycin (Kan) and 5-bromo-4-chloro-indolyl-β-D-galactopyranoside (X-gal). Hydrolysis of X-gal by functional β-galactosidase produces the characteristic blue color indicative of a Lac+ phenotype, whereas colonies that do not contain any functional β-galactosidase are white. Only white colonies are picked for subsequent study. Correct splicing is indicated by the precise removal of the genetically marked IVS and joining of the HBG (human β-globin) exon to the “captured” exon on an inserted fragment. This mode of exon capture coupled with next generation DNA sequencing can usefully identify exonic variants (SNPs, point mutations and indels) associated with a cancer genome. Short Oligonucleotide Analysis Package (SOAPalinger) can be employed for short oligonucleotide alignment to enable their assembly into longer DNA sequence reads capable of being mapped to the reference human genome using BLAST alignment tool together with the UCSC database to reveal sequence differences between tumor and control DNA specifically in their genic regions.

In summary of Example 2, an array of genic dense amplicons was produced by a polymerase chain reaction (“PCR”) process using an isolated genomic DNA and three inter-Alu consensus sequence-based primers. The PCR process comprised isolating genomic DNA from cells forming isolated genomic DNA. A PCR reaction mixture comprising:
(i) one or a plurality of Head-type Alu- or AluY- or any other Alu-subfamily-consensus sequence-based primer;
(ii) one or a plurality of Tail-type Alu- or AluY- or any other Alu-subfamily-consensus sequence-based primer;

(iii) the isolated genomic DNA; and
(iv) a PCR reaction mixture that contained the combination of an inter-Alu-PCR reaction combination, and PCR reaction mixture having a set of free deoxynucleotide triphosphate A,G,T and C nucleotides; a thermostable DNA polymerase; and a PCR buffer mixture.

The PCR reaction was completed using an inter-Alu PCR cycle program in a PCR machine that yielded the array of genic dense inter-Alu amplicons. This unique array of inter-Alu amplicons could be verified by visualizing a non-banded smear pattern on an electrophoretogram. Some experiments isolated genomic DNA from blood cells. Additionally, the three primers that were selected comprised:
SEQ ID No.: 2 (5′ TGGTCTCGAT CTCCTGACCT C-3) as the one Head-type inter AluY consensus sequence-based primer;
SEQ ID No.: 1 (5′GAGCGAGACTCCGTCTCA-3′) as the first Tail-type inter-AluY consensus sequence-based primer; and
SEQ ID No.: 6 (5′-AGCGAGACTCCG-3′) as the second Tail-type inter-AluY consensus sequence-based primers.

Moreover, a method for determining presence or absence of exotic variants in genomic DNA isolated from a cell was described. More specifically, the method utilized shotgun-cloning an array of genic dense amplicons produced by a three primer based inter-Alu region primer polymerase chain reaction (“PCR”) process into an adenovirus shuttle vector, to produce an array of inter-Alu-DNA plasmid vectors. The array of inter-Alu-DNA plasmid vectors was isolated and pooled and then transfected into a first retroviral packaging cell line that provides proteins useful for propagating the inter-Alu-DNA Plasmid vectors as an inter-Alu RNA-retroviruses. By promoting the retroviral packaging cell line to package both spliced and non-spliced inter-Alu-RNA-retroviruses into virons, an array of inter-Alu-RNA-virons could be produced and harvested. By infecting a second retroviral packaging cell line with the harvested inter-Alu-RNA virons from step an array of viral-inter-Alu stocks was produced, which were used to infect CV-1 cells (simian in Origin and are carrying a SV40 genetic material) or (“COS cells”) with at least one of the viral-inter-Alu stocks to provide circular inter-Alu-DNA episomes. These circular inter-Alu-DNA episomes were isolated from the COS cells and linearized with a restriction enzyme to form a linear-inter-Alu-DNA and used to transform bacterial cells with the linear-inter-Alu-DNA to create colony forming bacterial cells.

The colony forming bacterial cells could be visualized as either a blue-colony or a white-colony when cells were grown on agar plates containing kanamycin and 5-bromo-4-chloro-indolyl-β-D-galactopyranoside. More specifically, the determination of the presence or absence of the exotic variants in genomic DNA isolated from the white-colony bacterial cells from step was achieved by using next generation DNA sequencing and a short oligonucleotide analysis package for mapping the inter-Alu-exon amplicons with respect to a reference genome database.

The primers for this method utilized:

SEQ ID No.: 2 (5′ TGGTCTCGAT CTCCTGACCT C-3) as the one Head-type inter AluY consensus sequence-based primer;

SEQ ID No.: 1 (5′GAGCGAGACTCCGTCTCA-3′) as the Tail-type inter-AluY consensus sequence-based primer; and

SEQ ID No.: 6 (5′-AGCGAGACTCCG-3′) as the second Tail-type inter-AluY consensus sequence-based primers.

The method step of selecting the first retroviral packaging cell line to be ψ2. One example of this experiment visualized the single nucleotide polymorphisms (“SNP”) as the exotic variant. Additionally, a cancer cell was selected as the cell to isolate DNA, and even more specifically, DNA was isolated from a glioma cell.

### Example 3

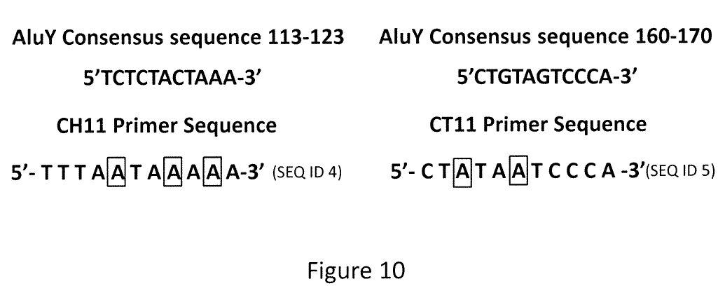

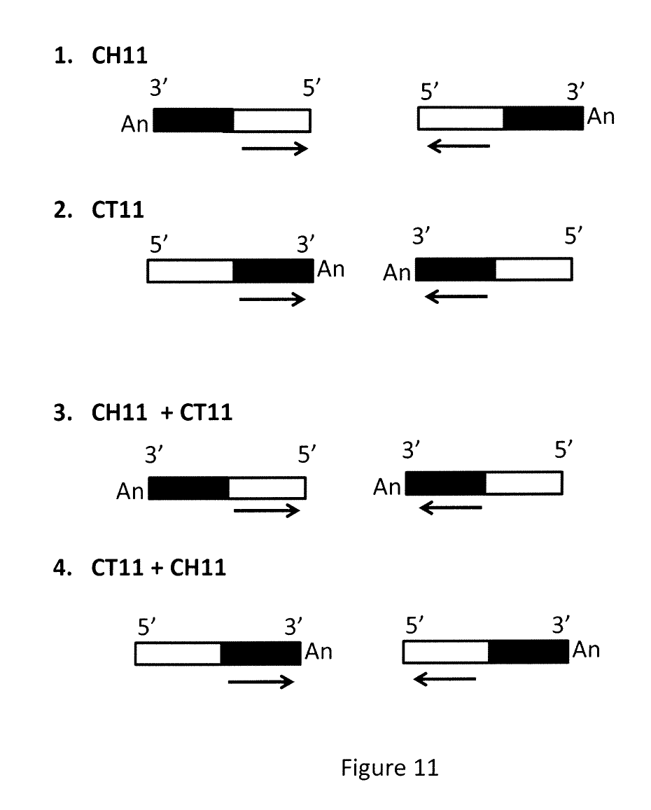

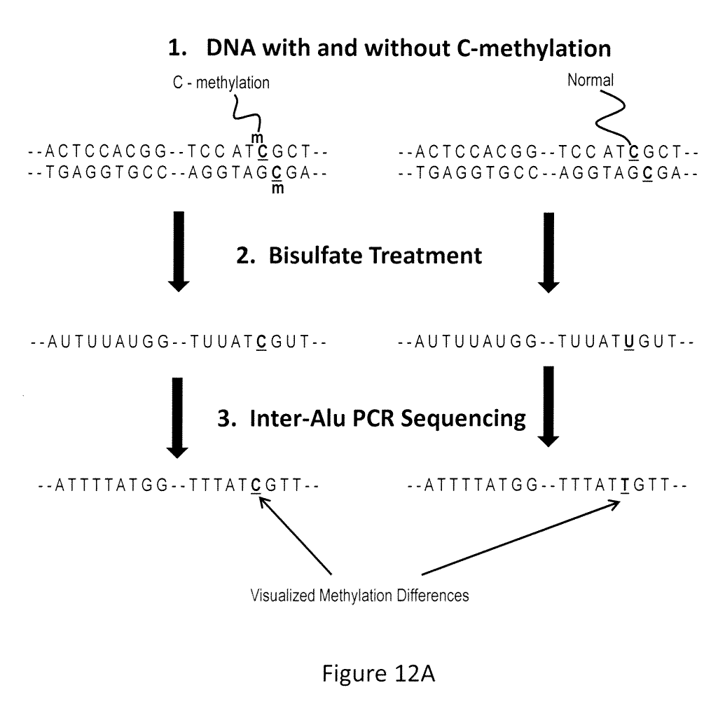

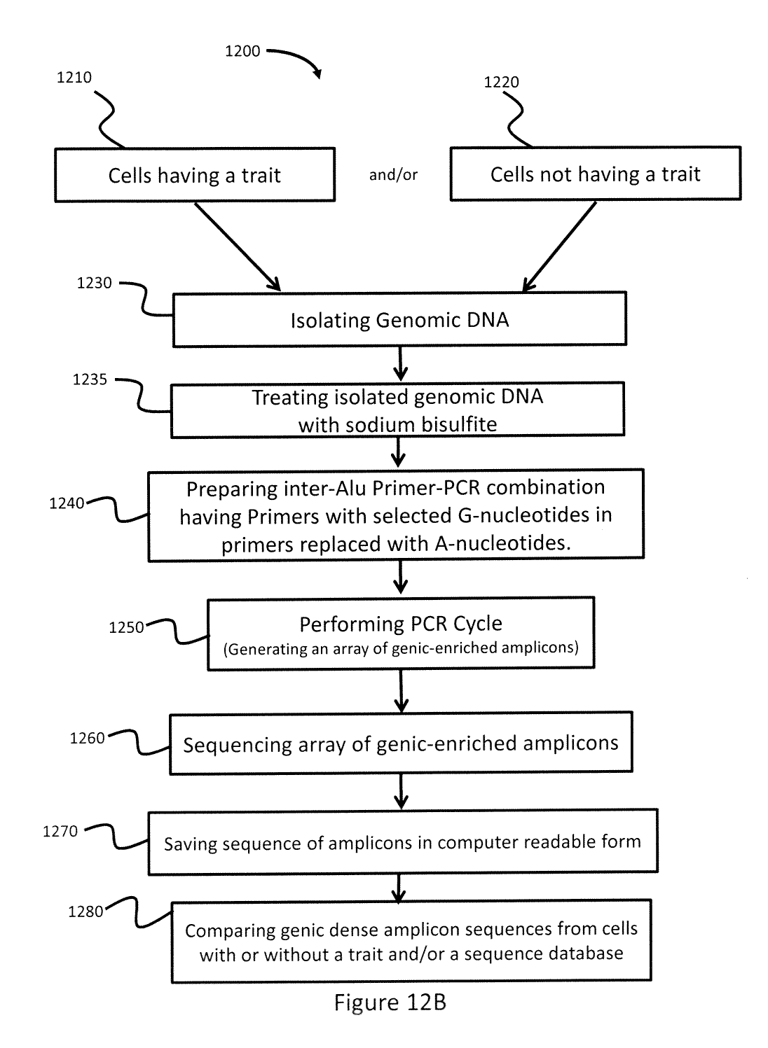

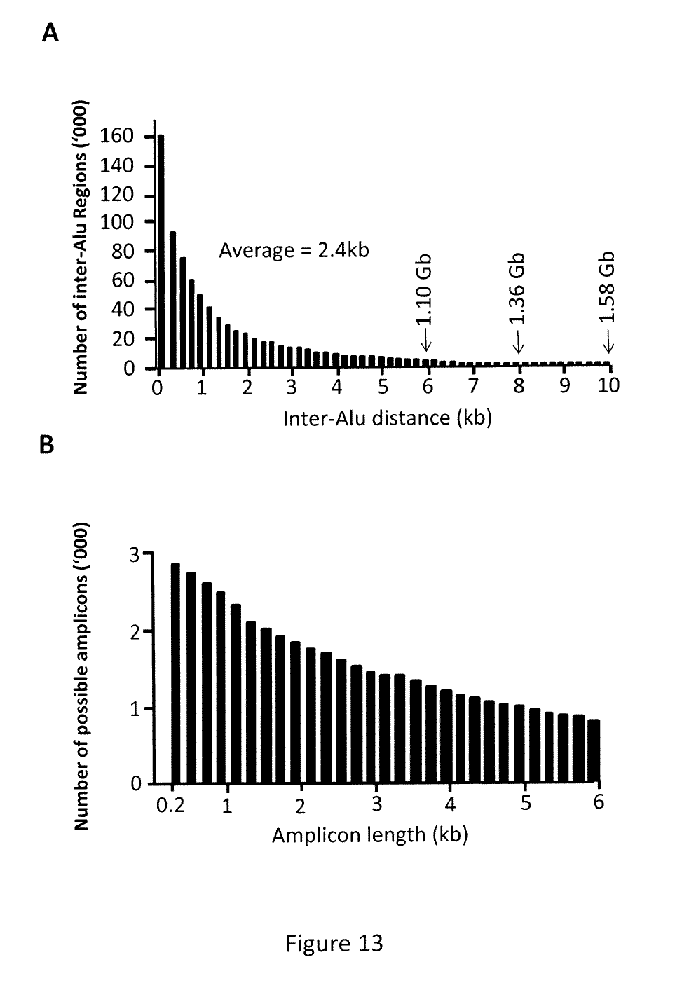

Example 3 illustrates the application of the present invention combining inter-Alu PCR and next generation sequencing to detect CpG methylations. Many 5′ mC are found within CpG dinucleotide-enriched Alu family repeats that make up 33% of the total CpG sites in the human genome. Previous studies have shown significant changes in the levels of CpG methylation in specific Alu sequences and their flanking regions in cancer and psychiatric disorders such as schizophrenia. This Embodiment describes the application of the present invention to asse the variation of CpG methylation in diseases. For this purpose, genomic DNA will be pretreated with bisulfite converting all unmethylated “C” including those at CpG sites to “T”. FIGS. 9-11 show two AluY consensus sequence-based PCR primers, viz. CT11 and CH11. CT11 is 11 bp long and a tail-type primer that can by itself in PCR generate inter-Alu sequences from one Alu 3′ tail to another Alu 5′ tail. CH11, also 11 bp long, is a head-type primer that can generate by itself inter-Alu sequences from one Alu 3′ head to another Alu 3′ head. When both CH11 and CT11 are added to the same inter-Alu PCR reaction, inter-Alu sequences from one Alu 5′ head to an adjacent Alu 3′ tail (head-to-tail direction), as well as from one Alu 3′ tail to an adjacent Alu 5′ head (tail-to-head direction) will also be obtained. Since all unmethylated “C” on the target genomic DNA would be converted to “T” by bisulfite treatment, CH11 and CT11 were designed such that the CT11 sequence corresponded to the complement of 160-170 bp of AluY consensus sequence, with all the “G” residues on the sequence replaced by “A”. Similarly, the CH11 sequence corresponded to the complement of segment 113-123 of AluY consensus sequence, with all the “G” residues converted to “A”.

In the inter-Alu PCR, 900 ng genomic DNA was incubated with 0.3M NaOH at 42° C. for 20 minutes, followed by 95° C. for 3 minutes and 0° C. for 1 minute. The DNA was then treated 2.0 M sodium bisulfite and 0.5 mM hydroquinone, topped with mineral oil and incubated at 55° C. for 16 hours. The bisulfite-treated DNA was purified, and amplified in inter-Alu PCR. Each PCR reaction had a final volume of 20 μl containing 4 μl 5× Mastermix (10×PCR buffer (500 mM KCl, 100 mM Tris-Cl, 15 mM MgCl2), 50 mM MgCl₂ and 10 mM dNTP mix), 1 μl 5 μM CH11 primer, 1 μl 5 μM CT11 primer, 0.1 μl thermostable DNA polymerase, 2 μl 10 ng/μl bisulfite-treated genomic DNA and 11.9 μl deionized water. PCR amplification included DNA denaturation at 95° C. for 5 min, followed by 20 cycles each of 30 s at 95° C., 30 s at 52° C., and 2 min at 72° C., plus finally another 5 min at 72° C. Because of the difficulty in amplifying bisulfite-treated genomic DNA by PCR, the steps described above were repeated once in order to enhance the quantity of amplicons. After completion of these PCR reactions, 5 μl PCR product were mixed with 50% glycerol, electrophoresed on 1.5% agarose gel, and inspected by UV visualization. Only 3 μg of the PCR amplified products containing Alu sequences and their flanking regions were required for the next-generation sequencing of the bisulfite treated DNA template sequences, where methylated “C” on the pre-treatment DNA would remain as “C” in the amplicons, whereas unmethylated “C” on the pre-treatment DNA would be converted to “T”. Following the sequencing, Short Oligonucleotide Analysis Package (SOAPalinger) was employed for short oligonucleotide alignment to assemble longer DNA sequence reads. BLAST alignment tool and UCSC database were employed to map these reads to the reference human genome to measure and compare the levels of methylation of CpG at specific sequence sites in tumor and control DNA. Besides cancer, Embodiment 3 can also be utilized in the measurement of DNA methylation levels at specific genomic CpG sites in a range of genetic diseases.

In summary of Example 3, an array of genic dense CpG methylated amplicons was produced using a polymerase chain reaction (“PCR”) process having an isolated genomic DNA treated with bisulfite and two modified inter-Alu consensus sequence-based primers. The PCR process utilized several steps, as indicated:
i) isolating genomic DNA from cells forming isolated genomic DNA;
ii) treating the isolated genomic DNA with sodium bisulfite for a first period of time, forming bisulfate-treated DNA;
iii) mixing a PCR reaction mixture comprising: (i) at least one Head-type inter-AluY consensus sequence-based primer; (ii) a first Tail-type inter-AluY consensus sequence-based primer; (iii) a second Tail-type inter-AluY consensus sequence-based primer; (iv) the isolated genomic DNA; and (v) a PCR reaction combination having a set of free deoxynucleotide triphosphate A, G, T and C nucleotides, a thermostable DNA polymerase, and a PCR buffer mixture; whereby the mixture comprises an inter-Alu-PCR reaction mixture;
iv) completing an inter-Alu PCR cycle program with the inter-Alu-PCR reaction mixture in a PCR machine and producing the array of genic dense CpG methylated amplicons.

The genomic DNA for Example 3 was isolated from normal or tumor cells. Additionally, the primers used included:
SEQ ID No.: 4 (CH11) 5′TTTAATAAAAA-3 as the first complement of the Head-Type inter-AluY consensus sequence-based primer having all G nucleotide residues replaced with A nucleotide residues; and
SEQ ID No.: 5 (CH11T) 5′-CTATAATCCCA-3′ as the first complement of the Tail-Type inter-AluY consensus sequence-based primer having all G nucleotide residues replaced with A nucleotide residues

The method for determining presence or absence of CpG variants in genomic DNA isolated from a diseased cell utilized the following steps:
isolating genomic DNA from cells forming isolated genomic DNA;
treating the isolated genomic DNA with sodium bisulfite for a first period of time, forming bisulfate-treated DNA;
mixing a PCR reaction mixture comprising: (i) a first complement of the head-type Alu consensus sequence-based primer having all G nucleotide residues replaced with A nucleotide residues; (ii) a first complement of the Tail-type Alu consensus sequence-based primer having all G nucleotide residues replaced with A nucleotide residues; (iii) a submicrogram level of the bisulfate-treated DNA; (iv) a mixture of free deoxynucleotide triphosphate A,G,T and C nucleotides; (v) a thermostable DNA polymerase; and (vi) a PCR buffer mixture, whereby the combination comprises an inter-Alu-PCR reaction mixture;
completing an inter-Alu PCR cycle program with the inter-Alu-PCR reaction mixture in a PCR machine and producing the array of genic dense CpG methylated amplicons; and
using next generation DNA sequencing and a short oligonucleotide analysis package for mapping the array of genic dense CpG methylated amplicons with respect to a reference genome database.

The isolated genomic DNA was selected from a normal and/or a tumor cell.

### Example 4

To complement next-generation sequencing technologies, there is a pressing need for efficient pre-sequencing capture methods with reduced costs and DNA requirement. The Alu family of short interspersed nucleotide elements is the most abundant type of transposable elements in the human genome and a recognized source of genome instability. With over one million Alu elements distributed throughout the genome, they are well positioned to facilitate genome-wide sequence amplification and capture of regions likely to harbor genetic variation hotspots of biological relevance, as exemplified in the previous examples.

Next-generation, massively-parallel sequencing technologies have transformed the landscape of genetics through their ability to produce giga-bases of sequence information in a single run. However, the sequencing cost, computation workload and amount of sample DNA required are still too high for large scale population analysis by means of whole-genome sequencing. There is clearly a need for pre-sequencing capture of subsets of the genome in order to reduce these requirements. Although the whole exome represents a valuable subset, its exclusion of introns, and the high cost and high DNA requirement for its analysis, remain major limitations. Other sequence subsets therefore clearly need to be explored.

Alu-transposons are a family of primate-specific short interspersed nucleotide elements (SINE) of ˜300 bp derived from 7SL RNA. Although Alu elements were once considered as ‘junk DNA’, their biological importance, in particular their influence on genome instability is being increasingly recognized. They are abundant in gene-rich regions, exert a major impact on genomic architecture, and increase local recombination rates. Previously we have found enhanced SNP frequencies in the vicinity of Alu-elements, more so among the youngest AluY elements than the intermediate-age AluS and the oldest AluJ. AluYs display also a higher rate of methylation, consistent with a stronger silencing pressure on these elements. Genotypic variations surrounding a human lineage-specific AluY insertion in the GABRB2 gene encoding GABAA receptor β2 subunit have been found by us to constitute a joint focal point for positive evolutionary selection, hotspot recombinations as well as association with schizophrenia and bipolar disorder. Neighborhoods of Alu-transposons are therefore a highly significant sequence subset of the human genome in terms of evolutionary development and pathogenesis.

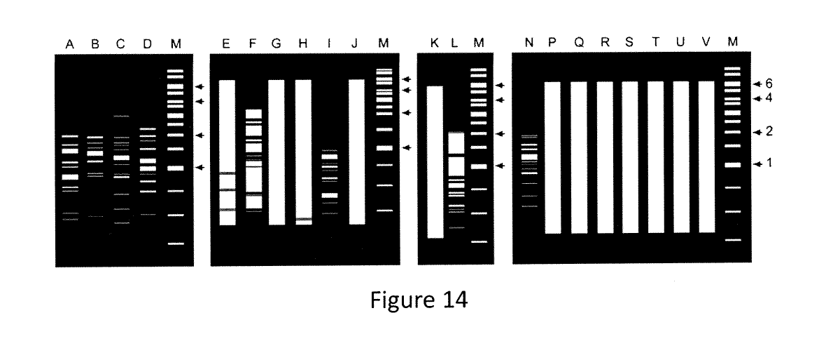

Inter-Alu PCR is a useful method for isolating human DNA in the presence of animal DNA, linkage mapping, creation of human specific probes and fingerprints, and detection of mutator phenotypes or high frequency genetic alterations. The general strategy of the method is to employ a single PCR primer based on the Alu consensus sequence to amplify the sequence between two Alu elements. With well over a million Alu-transposons in the human genome, the average distance between two Alus is only 2.4 kb (FIG. 14A), which suggests that inter-Alu PCR with an enhanced amplicon range coupled to next-generation sequencing could yield a huge sequence subset of the human genome for analysis. Accordingly the objective of the present study is to examine the possibility of enhancing the amplicon range of inter-Alu PCR and combining it with next generation sequencing to scan for sequence and structure variations in the human genome.

Here we report on the use of inter-Alu PCR with an enhanced range of amplicons in conjunction with next-generation sequencing to generate an Alu-anchored scan, or ‘AluScan’, of DNA sequences between Alu transposons, where Alu consensus sequence-based ‘H-type’ PCR primers that elongate outward from the head of an Alu element are combined with ‘T-type’ primers elongating from the poly-A containing tail to achieve huge amplicon range. To illustrate the method, glioma DNA was compared with white blood cell control DNA of the same patient by means of AluScan. The over 10 Mb sequences obtained, derived from more than 8,000 genes spread over all the chromosomes, revealed a highly reproducible capture of genomic sequences enriched in genic sequences and cancer candidate gene regions. Requiring only sub-micrograms of sample DNA, the power of AluScan as a discovery tool for genetic variations was demonstrated by the identification of 357 instances of loss of heterozygosity, 341 somatic indels, 274 somatic SNVs, and seven potential somatic SNV hotspots between control and glioma DNA.

Individual Alu-transposons in the human genome are on the average only 15-20% divergent from each other, and PCR primers complementary to the Alu consensus sequence have been employed for inter-Alu PCR. Likewise PCR primers based on consensus sequences in the AluJ, AluS and AluY subfamilies could also be devised. All Alu-based primers can be divided into ‘H-type’ where the primer extends outward from the head of the Alu, or ‘T-type’ where it extends outward from the poly-A containing tail. Previously, single general Alu consensus primers had given rise to agarose gel electrophoretograms displaying largely banded, banded plus smeared, or largely smeared patterns. In the present study, varying combinations of Alu, AluJ, AluS and/or AluY consensus primers were found to yield widely different electrophoretogram patterns. The presence of a single H-type or T-type primer tended to yield a banded, non-smeared pattern suggestive of a limited amplicon range (lanes A-D, FIG. 14). In lanes I and L respectively, even two or three T-type primers failed to give a non-banded pattern; lane K with three H-type primers gave a smeared pattern but lane F with two H-type primers gave only a banded pattern. In contrast, various primer combinations containing both H-type and T-type primers, allowing the amplification of intervening sequences between two Alu heads, between two Alu-tails as well as between one head and one tail, readily yielded a smeared gel indicating the presence of a wide diversity of amplicons of different sizes (lanes E, G, H, J, P-V). Therefore inclusion of both H-type and T-type primers provided the most reliable method for achieving huge amplicon range using no more than a small number of primers. The greater staining intensity of lane S compared to lane R further showed that the amounts of amplicons obtained from the same primer set could be increased by increasing the primer concentrations.

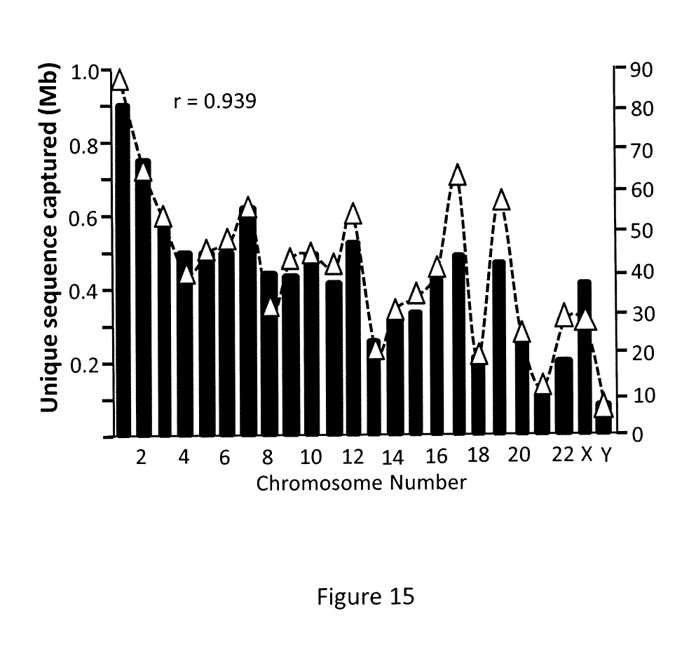

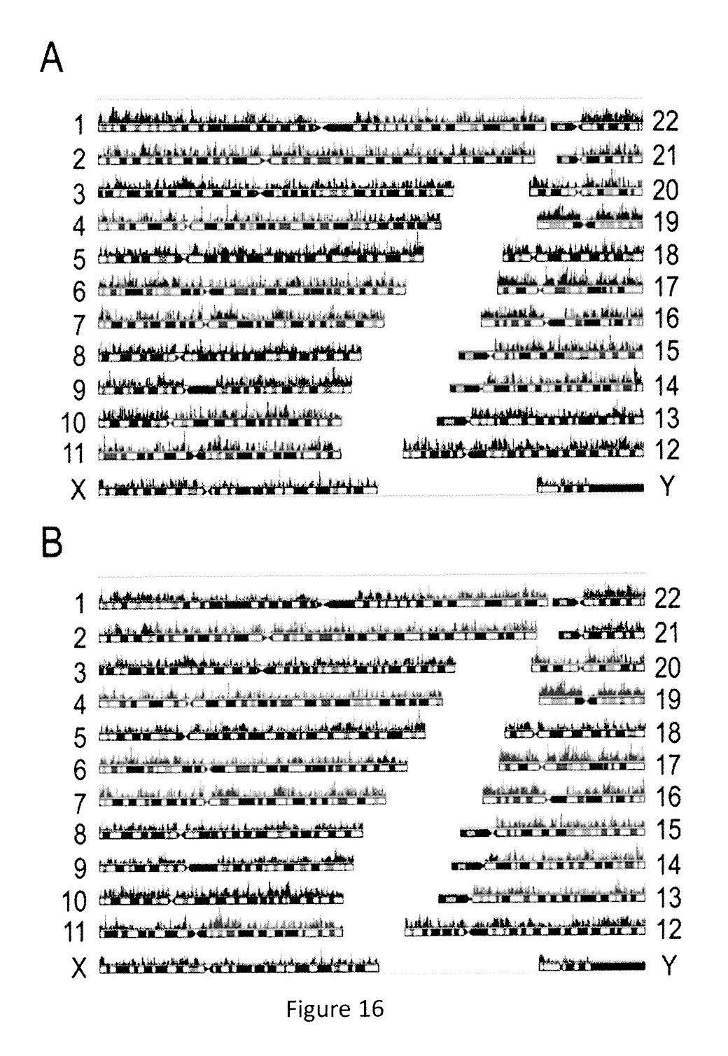

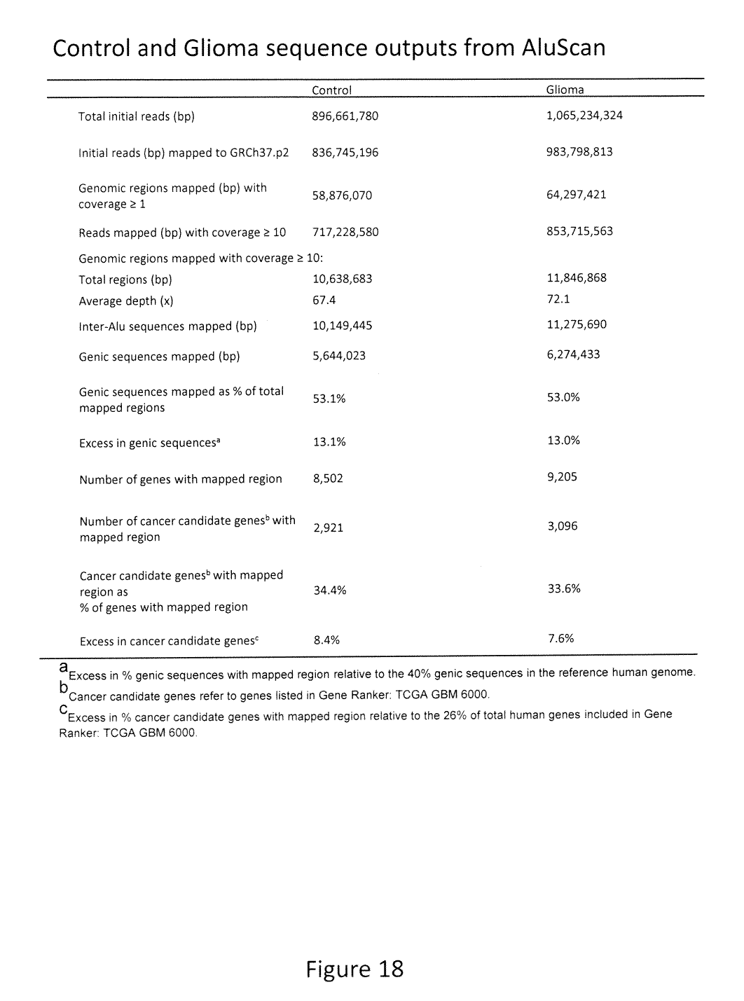

When AluScans were performed on paired control and cancer DNAs extracted from respectively the white blood cells and glioma tissue of a male Han Chinese patient using the three primers AluY278T18, AluY66H21 and R12A/267 described under Methods, smeared gels of amplicons up to ˜6 kb in size were obtained (FIG. 14, lane Q for control DNA). In each case the use of 90 ng sample DNA yielded sufficient amplicons for next-generation sequencing on the Illumina platform with a single flowcell lane and 75 bp paired-end reads. The sequencing output has been submitted to Sequence Read Archive (SRA) of NCBI. As indicated in FIG. 18, 837 Mb of the initial reads of control white blood cell DNA were mapped using the BWA program to 58.9 Mb regions on the reference human genome (GRCh37.p2), including high quality mapping of 717 Mb reads to 10.6 Mb regions with minimum 10 times and average 67 times coverage. Of the latter 10.6 Mb, 95% were inter-Alu sequences, which compared favorably with the NimbleGen SeqCap Exome array for targeted exon capture with typically 71% mapped reads on target; 53% were genic sequences including both exons and introns from 8,502 genes, representing an enrichment of genic sequences compared to the overall 40% gene content of the human genome; and 34% of the genes belonged to the list of cancer candidate genes in Gene Ranker: TCGA GBM 6000, exceeding the 26% of all human genes included in that list. The genomic regions mapped by the reads followed closely the number of Alu transposons located on the chromosomes (FIG. 15). With glioma DNA, 984 Mb of the initial reads were mapped to 64.3 Mb genomic regions, including 11.8 Mb high quality regions with minimum 10 times and average 72 times coverage. The overlap between the high quality regions mapped by the control- and glioma-reads totaled 9.5 Mb, equal to 90% of the control-mapped regions; the correlation in read coverage between control and glioma reads was high, with r=0.958; the density distributions of the control and glioma reads along different chromosomes (FIG. 16) were also highly correlated, with r=0.957. These results provided evidence that AluScan performed with the same set of primers enabled a reproducible genome-wide capture of DNA sequences that were enriched in both genic content and cancer candidate genes despite the many overlapping inter-Alu amplicons that might be amplified by a mixture of H- and T-type primers.

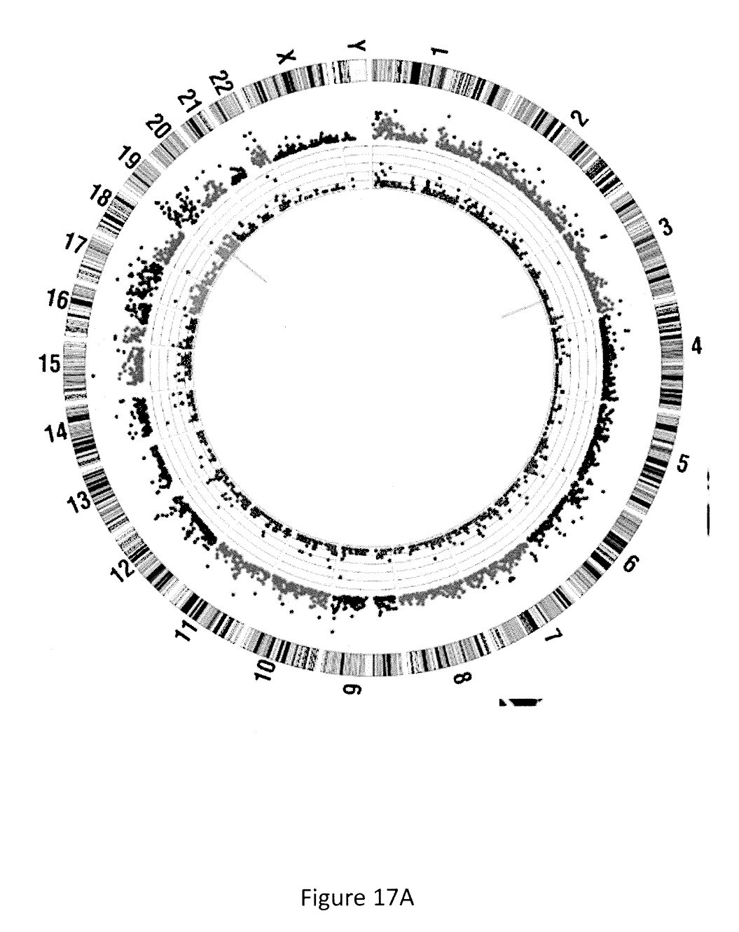

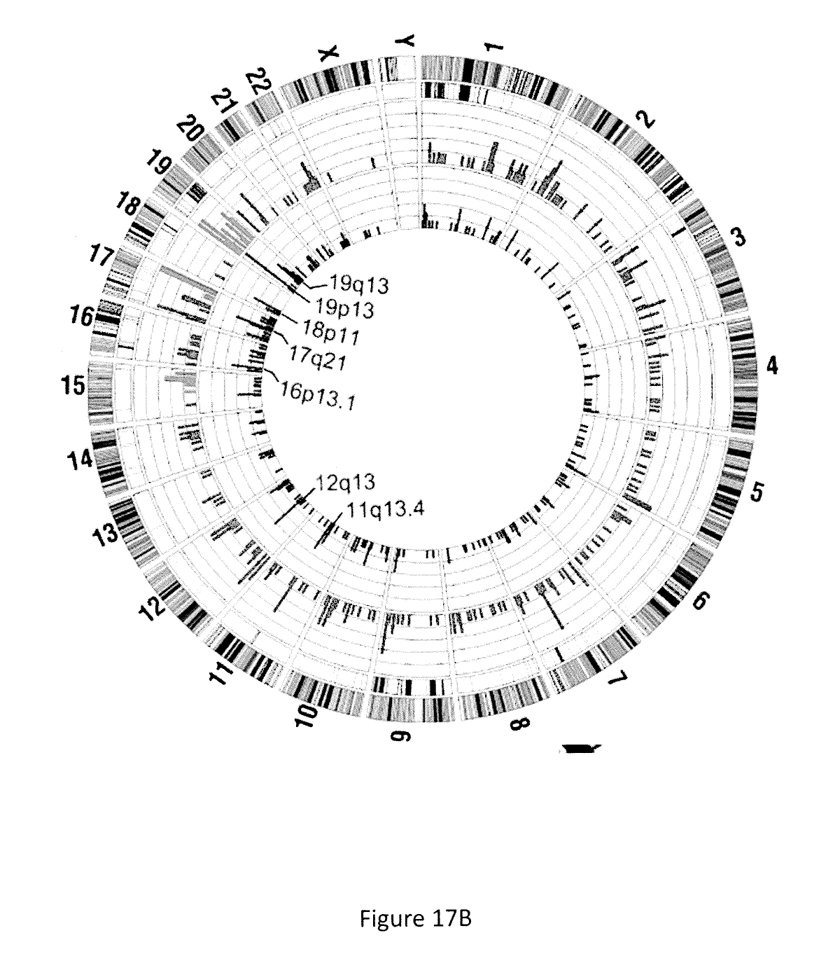

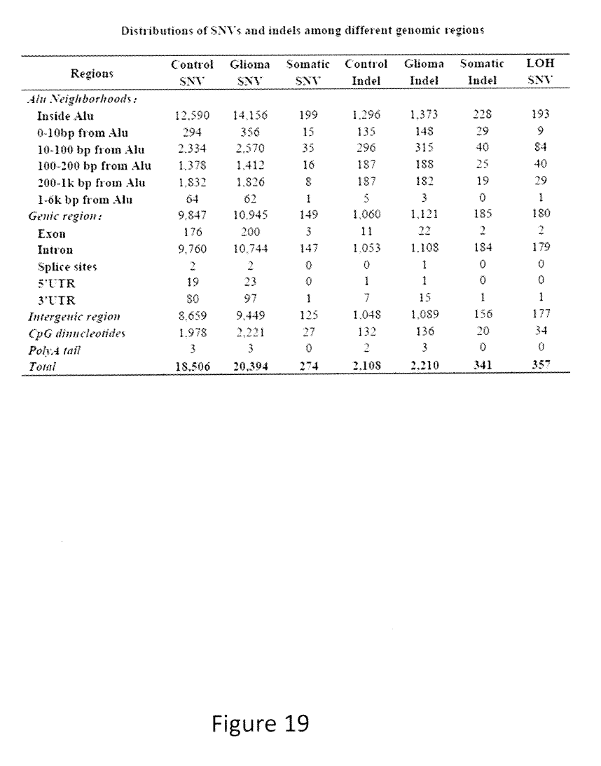

FIG. 17A and FIG. 19 show the distributions of genetic variations occurring in the 10.6 Mb control genomic sequences relative to reference human genome among different chromosomes and types of genomic regions: there were 18,506 germline SNVs, 11,039 or 59.6% of which were novel SNVs absent from dbSNP132, 2,108 small (≦30 bp) indels and two larger indels, viz. a 75-bp deletion on chromosome 19 and a 767-bp deletion on chromosome 3. When 60 SNV-containing genic segments including 10 segments each containing a novel SNV were randomly chosen from the control AluScan output for Sanger sequencing, the accuracy of successful SNV verification was 81.6%.

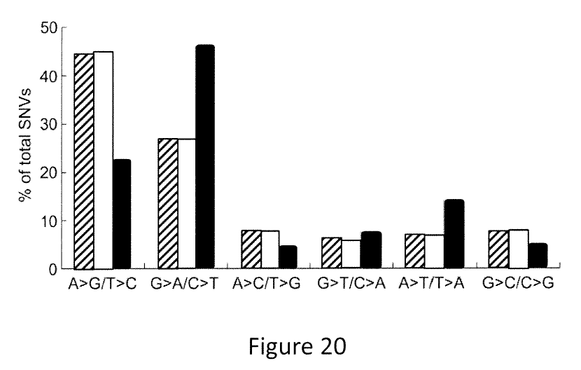

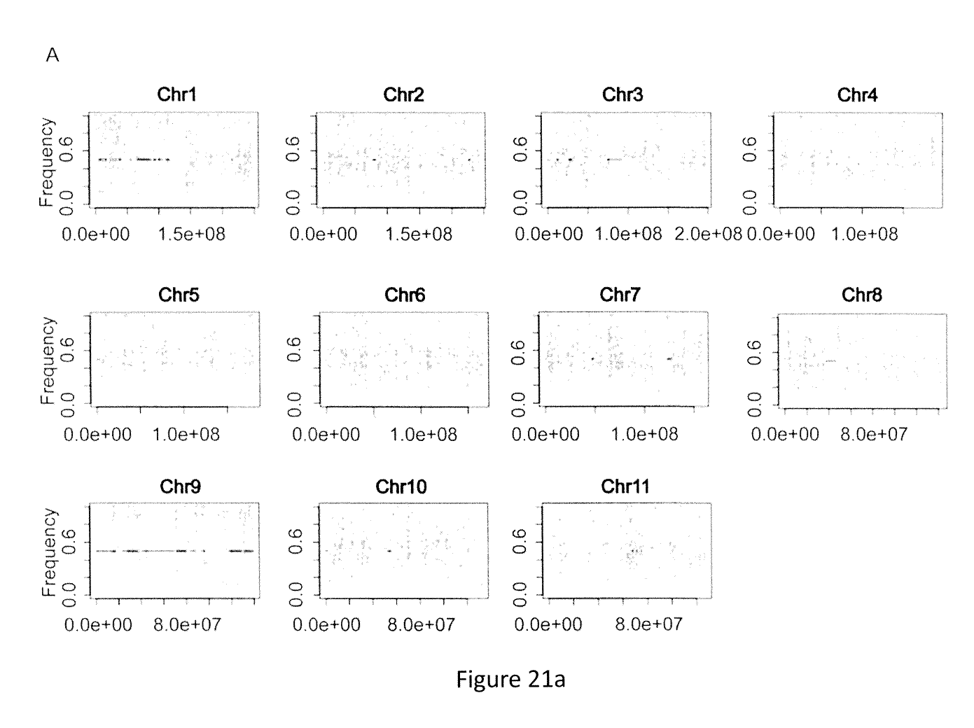

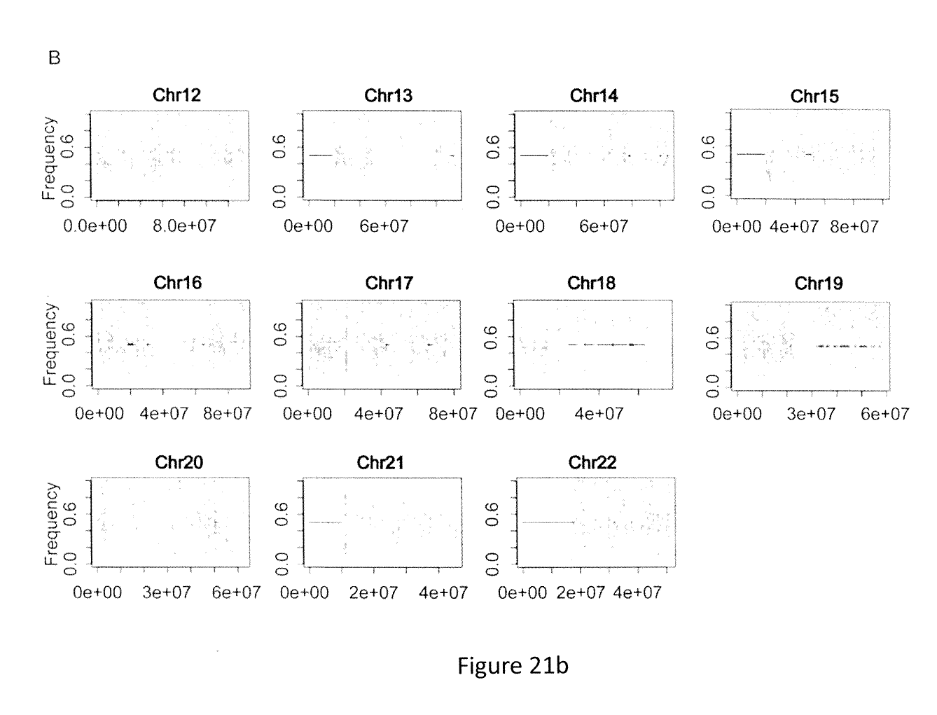

Comparison of the mapped control and glioma sequences identified 274 somatic SNVs between them, 70.4% of which represented novel SNVs absent from dbSNP132. In the control and glioma SNVs relative to the reference human genome, as well as the somatic SNVs occurring between control and glioma, transitions were far more numerous than transversions (FIG. 20). There were 357 instances of loss of heterozygosity (LOH) and 341 somatic indels between control and glioma DNAs. The LOHs were unequally distributed among different chromosomes (FIG. 21). Of the four particularly LOH-enriched regions, viz. regions 1p, 9p, 9q and 19q indicated in FIG. 17B notably 1p and 19q were known to contain glioma-associated deletions, which furnished valuable cross validation between AluScan and other genomic approaches.

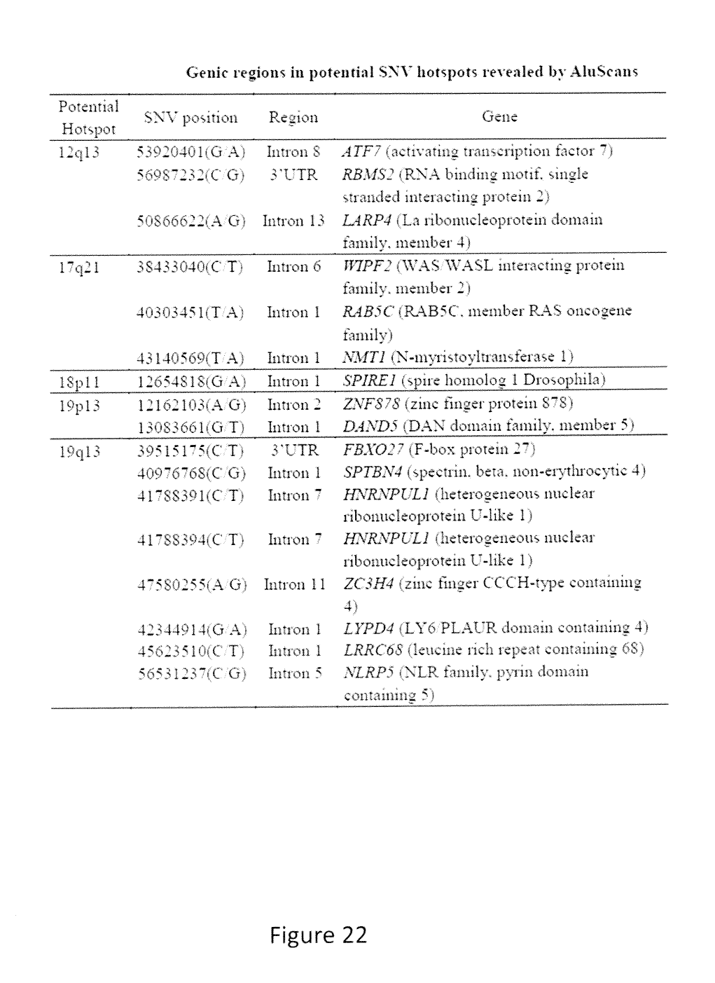

Seven 5-Mb intervals in the glioma sequences displayed enhanced numbers of somatic SNVs, where the number of somatic SNVs>4, indicating the potential presence of somatic SNV hotspots FIG. 17B. Of these seven potential SNV hotspots, those in chromosomal regions 12q13, 17q21, 18p11, 19p13 and 19q13 harboured altogether 16 SNV-containing genes including RAB5C of the RAS oncogene family in 17q21 (FIG. 22). None of these 16 genes were included in OMIM as a known glioma-associated gene. These findings illustrated the usefulness of AluScan as a discovery tool.

AluScan implemented with just a small number of H-type and T-type inter-Alu PCR primers, provides an effective capture of a diversity of genome-wide sequences for analysis. The method, by enabling an examination of gene-enriched regions containing exons, introns, and intergenic sequences with modest capture and sequencing costs, computation workload and DNA sample requirement is particularly well suited for accelerating the discovery of somatic mutations, as well as analysis of disease-predisposing germline polymorphisms, by making possible the comparative genome-wide scanning of DNA sequences from large human cohorts.

Using only 90 ng sample DNA in each instance, the AluScans performed in the present study with one H-type and two T-type primers generated reads that covered a total of ˜58-64 Mb, or ˜1.9-2.1% of genomic sequences. This total was comparable in order of magnitude to the genomic sequences in principle capturable by the set of three H and T-type consensual Alu-based primers employed, which were estimated to be ˜14 Mb for exact primer-template matches, or ˜106 Mb allowing for one mismatched base-pair per primer (FIG. 14B), but still far below the total of 1.10 Gb inter-Alu regions of ≦6 kb in length in the human genome (FIG. 14A). Thus there could be ample room for widening the scope of AluScan-capturable sequences through the use of diverse combinations of H- and T-type primers. Primers specific for other transposable elements such as LINEs, LTRs, as well as other types of more specialized primers could also be utilized to tailor the AluScan capture to a given investigational goal. Moreover, by treating target DNA with bisulfite to modify unmethylated C-residues prior to AluScan, epigenomic changes in normal and diseased cells may also be monitored.

By combining the twin advantages of multitudinous amplification of inter-Alu sequences through the joint usage of H-type and T-type primers, and massively parallel next-generation sequencing, AluScan thus provides a new method for genome-wide investigation in addition to whole genome sequencing (WGS) and whole exome sequencing (WES). WGS is the standard in comprehensiveness, but incurs high operation cost, large computation workload and multi-microgram DNA requirement. WES provides integral insight into the entire exome, but leaves the intronic regions uncharacterized, besides incurring high capture cost and multi-microgram DNA requirement. AluScan permits an examination of gene-enriched segments of exons, introns and intergenic sequences requiring comparatively modest capture and sequencing costs, lighter computation workload and only sub-microgram DNA samples. These three methods complement one another, together making possible a comprehensive analysis of sequence and structure variations of the human genome.

AluScan implemented with just a small number of PCR primers based on consensus Alu sequences provides a multiplex method for genome-wide sequence analysis. Through the inclusion of H and T type primers, the approach employs the abundance and wide distribution of Alu elements in the human genome as the basis for the effective capture of a huge number of DNA sequences in the vicinity of Alu elements. As demonstrated by the strong correlation between the captured white blood cell and glioma sequences, the same set of H and T-type primers has led to an extensively reproducible subset of genomic sequences in the two separate AluScans. As well, at least for this set of H and T-type primers, the captured sequences were enriched in genic and cancer-related DNA sequences.

The results in FIG. 17B illustrate the utility of AluScan as a discovery tool. Comparison of the paired while blood cell-glioma DNAs of a single patient has led to the uncovering of 357 LOHs and 274 somatic SNVs, a majority of which likely arising in the glioma, and seven potential SNV hotspots located on six different chromosomes. Importantly, the modest technical cost and DNA sample size required for AluScan will render practicable a follow up with similarly paired AluScans for tens to hundreds of glioma patients in order to distinguish the somatic and germline driver mutations fundamental to the development of the disease from passenger mutations. A major application of AluScan will thus reside in its facilitation of large cohort studies for clinical and biological investigations of the human genome.

Paired blood and cancer samples were obtained with consent and institutional ethics approval from a male Chinese Han patient with anaplastic oligodendroglioma at Beijing Tiantan Hospital for the preparation of control DNA by phenol-chloroform extraction and cancer genomic DNA using the AllPrep kit (Qiagen).

Inter-Alu PCR and next-generation sequencing. Fifteen parallel 25-μl PCR reaction mixtures each containing 2 μl Bioline 10× NH4 buffer (160 mM ammonium sulfate, 670 mM Tris-HCl, pH 8.8, 0.1% stabilizer), 3 mM MgCl₂, 0.15 mM dNTP mix, 0.3 μM AluY278T18 primer, 0.18 μM AluY66H21 primer, 0.06 μM R12A/267 primer, 1 unit Bioline Taq polymerase, and 6 ng control or glioma DNA. PCR amplification for AluScan included DNA denaturation at 95° C. for 5 min, followed by 35 cycles each of 30 s at 95° C., 30 s at 54° C., and 5 min at 71° C., and finally another 5 min at 71° C. Amplicons were purified with ethanol precipitation, and ≧3 μg purified products per sample were employed for Illumina GAII library construction and sequencing at Beijing Genomics Institute (Shenzhen, China). AluY278T18 (5′-GAGCGAGACTCCGTCTCA-3′) (SEQ ID 1), where ‘AluY’ represents the subfamily, ‘278’ the first position on the AluY consensus sequence paired with the primer, ‘T’ a ‘Tail-type’ primer (vs. ‘H’ for ‘H-type’), and ‘18’ the length of the primer, and AluY66H21 (5′-TGGTCTCGATCTCCTGACCTC-3′) (SEQ ID 2) were AluY consensus primers. R12A/267 (T-type) was an Alu consensus primer employed earlier for inter-Alu PCR at an annealing temperature of 56° C.

Agarose gel electrophoresis. PCR was performed basically as described in the preceding section, except that one PCR tube of 20 μl containing 100 ng control DNA was employed. The annealing temperatures were chosen to maximize in each instance the yield of amplicons: 60° C. for lane A in FIGS. 14,2, 58° C. for B-D, 56° C. for H and L, 64° C. for N, and 54° C. for the other lanes. Primer concentration was 0.30 μM for the single-primer lanes A-D; 0.15 μM per primer for the two-primer lanes E-J; 0.10 μM per primer for the three-primer lanes K, L, R, T-V; 0.30 μM per primer for the triple-dosed lane S. The concentrations of primers AluY278T18, AluY66H21 and R12A/267 in lane Q were 0.375 μM, 0.225 μM, 0.075 μM respectively; lane P was same as lane Q with omission of R12A/267; and lane N was same as lane P with further omission of AluY66H21.

Read mapping and variant analysis. Sequence reads were mapped to the GRCh37.p2 reference human genome using BWA (bwa-short algorithm version 0.5.9rc1) with default settings. Initial mapping results were transferred into indexed and sorted BAM format using SAMtools version 0.1.12a and further recalibrated and locally realigned using the Genome Analysis Toolkit (GATK version 1.0.4905) software. Regions with read depths of <10× were not analyzed further.

The UnifiedGenotyper module in GATK was used to produce the primary SNV calls, which were filtered using the parameter ‘-stand_call_conf 50.0’ and the Variant Filtration module, ensuring a coverage depth >10×, mapping quality >25.0 and strand bias <0. SNVs in the vicinity of indels were removed by means of the IndelGenotyperV2 module. Further filtration was achieved using the criterion that homozygous reference loci have a non-reference read frequency of <10%, heterozygous SNVs have a non-reference read frequency of ≧10% and <85%, and homozygous non-reference SNVs have a non-reference read frequency of ≧85%. Small indels were called using mpileup with ‘-ugf’ and bcftools with ‘-bvcg’ in SAMtools; and the calls were filtered using the script vcfutils.pl in SAMtools with default settings. Structural variants were identified initially using BreakDancer version 1.1 and refined using Pindel version 0.20. Somatic SNVs were defined as heterozygous loci present in the tumor genome that corresponded to homozygous loci in the control genome, and LOH SNVs were defined as heterozygous loci present in the control genome that corresponded to homozygous loci in the tumor genome. Novel somatic SNVs were obtained by removing all LOHs and those SNVs already reported in dbSNP132. LOHs were identified by comparison between control and glioma reads using ExomeCNV version 1.23.0.

### Example 5

One other aspect of the current invention is an array of inter-Alu gene-enriched amplicons produced by a polymerase chain reaction (“PCR”) process. The process of making the array comprises combining (a) a plurality of Alu consensus sequence-based primers with (b) a genomic DNA template isolated from cells; and (c) a PCR-extension mix. The PCR-extension mix comprises a set of free deoxynucleotide triphosphate A,G,T and C bases; a thermostable DNA polymerase; and a buffer solution to give an inter-Alu-PCR-mixture. The next step is completing an inter-Alu PCR cycle program with the inter-Alu-PCR-mixture in a PCR machine for a period of time to produce the array of inter-Alu gene-enriched amplicons.

Another aspect of the current invention involves selecting the plurality of Alu consensus sequence-based primers to be AluY consensus sequence based primers from the AluY subfamily of Alu elements. A step of selecting the plurality of Alu consensus sequence-based primers to be: a Head-type AluY consensus sequence-based primer; a First Tail type AluY consensus sequence based primer; and a Second tail type AluY consensus sequence based primer, is also visualized in the present invention. A first specific example of the PCR process of described above using SEQ ID No.: 2 (5′ TGGTCTCGAT CTCCTGACCT C-3) as the Head-type AluY consensus sequence-based primer; selecting SEQ ID No.: 1 (5′GAGCGAGACT CCGTCTCA-3′) (AluY278T18) as the first Tail-type AluY consensus sequence-based primer; and selecting SEQ ID No.: 6 (5′-AGCGAGACTCCG-3′) (R12A/267) as the general Alu consensus sequence-based primer.

A second specific example of the PCR process of described above using SEQ ID No.: 7 (5′ GGCTCAAGCG ATCCTC 3′) (AluJo56H16) as the Head-type AluJo consensus sequence-based primer; selecting SEQ ID No.: 9 (5′ ACCTCAGGTG ATCCAC 3′) (AluSq56H16) as the Head-type AluSq consensus sequence-based primer; and selecting SEQ ID No.: 6 (5′-AGCGAGACTCCG-3′) (R12A/267) as the general Alu consensus sequence-based primers.

A third specific example of the PCR process of described above using SEQ ID No.: 7 (5′ GGCTCAAGCG ATCCTC 3′) (AluJo56H16) as the Head-type AluJo consensus sequence-based primer; selecting SEQ ID No.: 9 (5′ ACCTCAGGTG ATCCAC 3′) (AluSq56H16) as the Head-type AluSq consensus sequence-based primer; and selecting SEQ ID No.: 10 (5′ AACAAGAGCG AAACTC 3′) (AluSq263T16) as the Tail-type AluSq consensus sequence-based primers.

A fourth specific example of the PCR process of described above usingSEQ ID No.: 7 (5′ GGCTCAAGCG ATCCTC 3′) (AluJo56H16) as the Head-type AluJo consensus sequence-based primer; selecting SEQ ID No.: 6 (5′-AGCGAGACTCCG-3′) (R12A/267) as the Tail-type general Alu consensus sequence-based primer; and selecting SEQ ID No.: 10 (5′ AACAAGAGCG AAACTC 3′) (AluSq263T16) as the Tail-type AluSq consensus sequence-based primers.

A fifth specific example of the PCR process of described above usingSEQ ID No.: 7 (5′ GGCTCAAGCG ATCCTC 3′) (AluJo56H16) as the Head-type AluJo consensus sequence-based primer; selecting SEQ ID No.: 8 (5′ TATGATCGTG CCACTG 3′) (AluJo232T16) as the Tail-type AluJo consensus sequence-based primer; and selecting SEQ ID No.: 10 (5′ AACAAGAGCG AAACTC 3′) (AluSq263T16) as the Tail-type AluSq consensus sequence-based primers. The PCR process as described above can utilize genomic DNA template from blood cells. Additionally, verifying the array of inter-Alu gene-enriched amplicons is accomplished by visualizing a non-banded smear pattern on an electrophoretogram.

A second aspect of the current invention involves a method of identifying relevant-inter-Alu-genetic elements associated with a trait. The method comprises: (a) selecting a paired set of discovery samples, wherein the trait is expressed in the first set of the paired array and not the second set of the paired array; (b) generating an array of inter-Alu-genic-elements for each of the paired set of discovery samples; (c) sequencing the array of inter-Alu-genic-elements for each of the paired set of discovery samples using a next generation sequencing technique forming a next generation inter-Alu sequencing pattern; (d) transforming a next generation inter-Alu sequencing pattern for each paired set of discovery samples in a computer readable format;

(e) identifying relevant-inter-Alu-genetic-elements from the next generation inter-Alu sequencing pattern when the quality of an identifiable genetic variations between the paired set of discovery samples is present, wherein the relevant-inter-Alu-element is identified as being associated with the trait. There are several variations of identifying relevant inter Alu genic elements as shown below:
identifying relevant-inter-Alu-genetic-elements when the next generation inter-Alu sequencing patterns have the quality of being present in a threshold percentage of sequence reads.
identifying relevant-inter-Alu-genetic-elements when the next generation inter-Alu sequencing patterns have the quality of being represented by a threshold read quality score at variant base(s).
identifying relevant-inter-Alu-genetic-elements when the next generation inter-Alu sequencing patterns have the quality of being present in sequence reads in a threshold number of strands.
identifying relevant-inter-Alu-genetic-elements when the next generation inter-Alu sequencing patterns have the quality of being present in sequence reads in a threshold number of bases.
identifying relevant-inter-Alu-genetic-elements when the next generation inter-Alu sequencing patterns have the quality of being present in sequence reads in a threshold number of regions.
identifying relevant-inter-Alu-genetic-elements when the next generation inter-Alu sequencing patterns have the quality of being aligned at a threshold level to a first reference sequence.
identifying relevant-inter-Alu-genetic-elements when the next generation inter-Alu sequencing patterns have the quality of being aligned at a threshold level compared with to a second reference sequence.
identifying relevant-inter-Alu-genetic-elements when the next generation inter-Alu sequencing patterns have the quality of being variant-genetic elements that do not have biasing features bases within a threshold number of nucleotides of the variant.
associating the relevant-inter-Alu genetic elements with the trait when the next generation inter-Alu sequencing patterns have the quality of being aligned at a threshold value of importance to a homeostasis marker of the trait.
associating the relevant-inter-Alu genetic elements with the trait when the next generation inter-Alu sequencing patterns have the quality of being aligned at a threshold value of trait severity.
associating the relevant-inter-Alu genetic elements with the trait when the next generation inter-Alu sequencing patterns have the quality of being aligned at a threshold value of an age of onset of the trait.
associating the relevant-inter-Alu genetic elements with the trait when the next generation inter-Alu sequencing patterns when the relevant-inter-Alu-genetic elements have the quality of being aligned at a specificity to the trait.
selecting the paired set of discovery samples from an individual having cell types with different traits.
selecting the paired set of discovery samples from a pedigree with different traits.
selecting the paired set of discovery samples from a subset of a single cohort with different traits.
selecting the paired set of discovery samples from a subset of a cohort with different traits.
selecting relevant-inter-Alu-genetic elements to be a loss of heterozygosity.
selecting relevant-inter-Alu-genetic elements to be a somatic indel, wherein the somatic indel is an insertion or deletion of genetic information.
selecting relevant-inter-Alu-genetic elements to be a small nucleotide variation (“SNV”).
selecting relevant-inter-Alu-genetic elements to be a CpG loci DNA methylation.
selecting relevant-inter-Alu-genetic elements to be a frameshift variant.
selecting the association of the relevant-inter-Alu element with the relevant-inter-Alu-component phenotype to be identified by a threshold value of the coincidence of the relevant-inter-Alu element and the relevant-inter-Alu component phenotype within a set of discovery samples.
selecting the set of discovery samples to include both affected samples and unaffected samples, wherein affected samples are samples associated with the relevant-inter-Alu component phenotype, wherein unaffected samples are samples not associated with the relevant-inter-Alu component phenotype.
selecting the trait to be a disease, a phenotype, a quantitative or qualitative trait, a disease outcome, or a disease susceptibility.
selecting the trait to be tumorigenic.
selecting the relevant-inter-Alu element further to be an element associated with one or more genetic elements associated with the trait.

### REFERENCES CITED

The following references, to the extent that they provide exemplary procedural or other details supplementary to those set forth herein are specifically incorporated herein by reference.

### U.S. Patent Documents

U.S. Pat. No. 7,537,889 Issued May 26, 2009, entitled “ASSAY FOR QUANTITATION OF HUMAN DNA USING ALU ELEMENTS,” Sinha, et al.
U.S. Pat. No. 5,773,649 Issued Jun. 30, 1998, entitled “DNA MARKERS TO DETECT CANCER CELLS EXPRESSING A MUTATOR PHENOTYPE AND METHOD OF DIAGNOSIS OF CANCER CELL,” Sinnett, et al.

### Non-Patent References

Ullu E, Tschudi C: Alu sequences are processed 7SL RNA genes. Nature 1984, 312:171-172.
Konkel M K, Batzer M A: A mobile threat to genome stability: The impactof non-LTR retrotransposons upon the human genome. Semin Cancer Biol 2010, 20:211-221.
Zhang Y, Romanish M T, Mager D L: Distributions of transposable elements reveal hazardous zones in mammalian introns. PLoS Comput Biol 2011, 7: e1002046.
Batzer M A, Deininger P L: Alu repeats and human genomic diversity. Nat Rev Genet. 2002, 3:370-379.
Lander E S, Linton L M, Birren B, Nusbaum C, Zody M C, Baldwin J. Devon K, Dewar K, Doyle M, FitzHugh W, Funke R, Gage D, Harris K, Heaford A, Howland J, Kann L. Lehoczky J, LeVine R, McEwan P, McKernan K, Meldrim J, Mesirov J P, Miranda C, Morris W, Naylor J. Raymond C, Rosetti M, Santos R, Sheridan A, Sougnez C, et al: Initial sequencing and analysis of the human genome. Nature 2001, 409:860-921.
Deininger P L, Moran J V, Batzer M A, Kazazian H H Jr: Mobile elements and mammalian genome evolution. Curr Opin Genet Dev 2003, 13:651-658.
Witherspoon D J, Watkins W S, Zhang Y, Xing J, Tolpinrud W L, Hedges D J, Batzer M A, Jorde L B: Alu repeats increase local recombination rates. BMC Genomics 2009, 10:530.
Ng S K, Xue H: Alu-associated enhancement of single nucleotide polymorphisms in the human genome. Gene 2006, 368:110-116.
Rodriguez J, Vives L, Jorda M, Morales C, Munoz M, Vendrell E, Peinado M A: Genome-wide tracking of unmethylated DNA Alu repeats in normal and cancer cells. Nucleic Acids Res 2008, 36:770-784.
Lo W S, Xu Z, Yu Z, Pun F W, Ng S K, Chen J, Tong K L, Zhao C, Xu X, Tsang S Y, Harano M, Stober G, Nimgaonkar V L, Xue H: Positive selection within the Schizophrenia-associated GABAA receptor β2 gene. PLoS One 2007, 2:e462.
Ng S K, Lo W S, Pun F W, Zhao C, Yu Z, Chen J, Tong K L, Xu Z, Tsang S Y, Yang Q, Yu W, Nimgaonkar V, Stober G, Harano M, Xue H: A recombination hotspot in a schizophrenia-associated region of GABRB2. PLoS One 2010, 5:e9547.
Lo W S, Lau C F, Xuan Z, Chan C F, Feng G Y, He L, Cao Z C, Liu H, Luan Q M, Xue H: Association of SNPs and haplotypes in GABAA receptor β2 gene with schizophrenia. Mol Psychiatry 2004, 9:603-608.
Zhao C, Xu Z, Wang F, Chen J, Ng S K, Wong P W, Yu Z, Pun F W, Ren L, Lo W S, Tsang S Y, Xue Alternative-splicing in the exon-10 region of GABAA receptor β2 subunit gene: relationships between novel isoforms and psychotic disorders. PLoS One 2009, 4:e6977.
Nelson D L, Ledbetter S A, Corbo L, Victoria M F, Ramirez-Solis R, Webster T D, Ledbetter D H, Caskey C T: Alu polymerase chain reaction: a method for rapid isolation of human-specific sequences from complex DNA sources. Proc Natl Acad Sci USA 1989, 86:6686-6690.
Zietkiewicz E, Labuda M, Sinnett D, Glorieux F H, Labuda D: Linkage mapping by simultaneous screening of multiple polymorphic loci using Alu oligonucleotide-directed PCR. Proc Natl Acad Sci USA 1992, 89:8448-8451.
Kass D H, Batzer M A: Inter-Alu polymerase chain reaction: advancements and applications. Anal Biochem 1995, 228:185-193.
Krajinovic M, Richer C, Labuda D, Sinnett D: Detection of a mutator phenotype in cancer cells by inter-Alu polymerase chain reaction. Cancer Res 1996, 56:2733-2737.
Srivastava T, Seth A, Datta K, Chosdol K, Chattopadhyay P, Sinha S: Inter-alu PCR detects high frequency of genetic alterations in glioma cells exposed to sub-lethal cisplatin. Int. J Cancer 2005, 117:683-689.
Li H, Durbin R: Fast and accurate short read alignment with Burrows-Wheeler transform. Bioinformatics 2009, 25:1754-1760.
Kim D W, Nam S H, Kim R N, Choi S H, Park H S: Whole human exome capture for high-throughput sequencing. Genome 2010, 53:568-574.
Durand K S, Guillaudeau A, Weinbreck N, DeArmas R, Robert S, Chaunavel A, Pommepuy I, Bourthoumieu S, Caire F, Sturtzand F G, Labrousse F J: 1p19q LOH patterns and expression of p53 and Olig2 in gliomas: relation with histological types and prognosis. Mol Pathol 2010, 23:619-628.
Park E S, Huh J W, Kim T H, Kwak K D, Kim W, Kim H S: Analysis of newly identified low copy AluYj subfamily. Genes Genet Syst 2005, 80:415-422.
Price A L, Eskin E, Pevzner P A: Whole-genome analysis of Alu repeat elements reveals complex evolutionary history. Genome Res 2004, 14:2245-2252.
Li H, Handsaker B, Wysoker A, Fennell T, Ruan J, Horner N, Marth G, Abecasis G, Durbin R: The Sequence Alignment/Map format and SAMtools. Bioinformatics 2009, 25:2078-2079.
McKenna A, Hanna M. Banks E, Sivachenko A, Cibulskis K. Kernytsky A, Garimella K, Altshuler D, Gabriel S, Daly M, DePristo M A: The Genome Analysis Toolkit: a MapReduce framework for analyzing next-generation DNA sequencing data. Genome Res 2010, 20:1297-1303.
Chen K, Wallis J W, McLellan M D, Larson D E, Kalicki J M, Pohl C S, McGrath S D, Wendl M C, Zhang Q, Locke D P, Shi X, Fulton R S, Ley T J, Wilson R K, Ding L, Mardis E R: BreakDancer: an algorithm for high-resolution mapping of genomic structural variation. Nat Methods 2009, 6:677-681.
Ye K, Schulz M H, Long Q, Apweiler R, Ning Z: Pindel: a pattern growth approach to detect break points of large deletions and medium sized insertions from paired-end short reads. Bioinformatics 2009, 25:2865-2871.
Sathirapongsasuti J F, Lee H, Horst B A, Brunner G, Cochran A J, Binder S, Quackenbush J, Nelson S F: Exome sequencing-based copy-number variation and loss of heterozygosity detection: ExomeCNV. Bioinformatics 2011, 27:2648-2654.
Krzywinski M, Schein J, Birol I, Connors J, Gascoyne R, Horsman D, Jones S J, Marra M A: Circos: an information aesthetic for comparative genomics. Genome Res 2009, 19:1639-1645.

What is claimed is:

1. An array of inter-Alu gene-enriched amplicons produced by a polymerase chain reaction (“PCR”) process comprising:
(a) combining:
i. a plurality of Alu consensus sequence-based primers
ii. a genomic DNA template isolated from cells; and
iii. a PCR-extension mix comprising:
a set of free deoxynucleotide triphosphate A,G,T and C bases;
a thermostable DNA polymerase; and
a buffer solution;
to give an inter-Alu-PCR-mixture,
(b) completing an inter-Alu PCR cycle program with the inter-Alu-PCR-mixture in a PCR machine for a period of time to produce the array of inter-Alu gene-enriched amplicons.

2. The PCR process of claim 1, further comprising a step of selecting the plurality of Alu consensus sequence-based primers to be AluY consensus sequence based primers from the AluY subfamily of Alu elements.

3. The PCR process of claim 1, further comprising a step of selecting the plurality of Alu consensus sequence-based primers to be: a Head-type AluY consensus sequence-based primer; a First Tail type AluY consensus sequence based primer; and a Second tail type AluY consensus sequence based primer.

4. The PCR process of claim 3, further comprising the step of selecting SEQ ID No.: 2 (5′ TGGTCTCGAT CTCCTGACCT C-3) as the Head-type AluY consensus sequence-based primer; selecting SEQ ID No.: 1 (5′GAGCGAGACT CCGTCTCA-3′) (AluY278T18) as the first Tail-type AluY consensus sequence-based primer; and selecting SEQ ID No.: 6 (5′-AGCGAGACTCCG-3′) (R12A/267) as the general Alu consensus sequence-based primer.

5. The PCR process of claim 3, further comprising the step of selecting SEQ ID No.: 7 (5′ GGCTCAAGCG ATCCTC 3′) (AluJo56H16) as the Head-type AluJo consensus sequence-based primer; selecting SEQ ID No.: 9 (5′ ACCTCAGGTG ATCCAC 3′) (AluSq56H16) as the Head-type AluSq consensus sequence-based primer; and selecting SEQ ID No.: 6 (5′-AGCGAGACTCCG-3′) (R12A/267) as the general Alu consensus sequence-based primers.

6. The PCR process of claim 3, further comprising the step of selecting SEQ ID No.: 7 (5′ GGCTCAAGCG ATCCTC 3′) (AluJo56H16) as the Head-type AluJo consensus sequence-based primer; selecting SEQ ID No.: 9 (5′ ACCTCAGGTG ATCCAC 3′) (AluSq56H16) as the Head-type AluSq consensus sequence-based primer; and selecting SEQ ID No.: 10 (5′ AACAAGAGCG AAACTC 3′) (AluSq263T16) as the Tail-type AluSq consensus sequence-based primers.

7. The PCR process of claim 3, further comprising the step of selecting SEQ ID No.: 7 (5′ GGCTCAAGCG ATCCTC 3′) (AluJo56H16) as the Head-type AluJo consensus sequence-based primer; selecting SEQ ID No.: 6 (5′-AGCGAGACTCCG-3′) (R12A/267) as the Tail-type general Alu consensus sequence-based primer; and selecting SEQ ID No.: 10 (5′ AACAAGAGCG AAACTC 3′) (AluSq263T16) as the Tail-type AluSq consensus sequence-based primers.

8. The PCR process of claim 3, further comprising the step of selecting SEQ ID No.: 7 (5′ GGCTCAAGCG ATCCTC 3′) (AluJo56H16) as the Head-type AluJo consensus sequence-based primer; selecting SEQ ID No.: 8 (5′ TATGATCGTG CCACTG 3′) (AluJo232T16) as the Tail-type AluJo consensus sequence-based primer; and selecting SEQ ID No.: 10 (5′ AACAAGAGCG AAACTC 3′) (AluSq263T16) as the Tail-type AluSq consensus sequence-based primers.

9. The PCR process of claim 1, further comprising the step of isolating the genomic DNA template from blood cells.

10. The PCR process of claim 1, further comprising the step of: (c) verifying the array of inter-Alu gene-enriched amplicons by visualizing a non-banded smear pattern on an electrophoretogram.

11. A method of identifying relevant-inter-Alu-genetic elements associated with a trait, the method comprising:
(a) selecting a paired set of discovery samples, wherein the trait is expressed in the first set of the paired array and not the second set of the paired array;
(b) generating an array of inter-Alu-genic-elements for each of the paired set of discovery samples;
(c) sequencing the array of inter-Alu-genic-elements for each of the paired set of discovery samples using a next generation sequencing technique forming a next generation inter-Alu sequencing pattern;
(d) transforming a next generation inter-Alu sequencing pattern for each paired set of discovery samples in a computer readable format;
(e) identifying relevant-inter-Alu-genetic-elements from the next generation inter-Alu sequencing pattern when the quality of an identifiable genetic variations between the paired set of discovery samples is present;
wherein the relevant-inter-Alu-element is identified as being associated with the trait.

12. The method of claim 11, further comprising the step of: (f) identifying relevant-inter-Alu-genetic-elements when the next generation inter-Alu sequencing patterns have the quality of being present in a threshold percentage of sequence reads.

13. The method of claim 11, further comprising the step of: (f) identifying relevant-inter-Alu-genetic-elements when the next generation inter-Alu sequencing patterns have the quality of being represented by a threshold read quality score at variant base(s).

14. The method of claim 11, further comprising the step of: (f) identifying relevant-inter-Alu-genetic-elements when the next generation inter-Alu sequencing patterns have the quality of being present in sequence reads in a threshold number of strands.

15. The method of claim 11, further comprising the step of: (f) identifying relevant-inter-Alu-genetic-elements when the next generation inter-Alu sequencing patterns have the quality of being present in sequence reads in a threshold number of bases.

16. The method of claim 11, further comprising the step of: (f) identifying relevant-inter-Alu-genetic-elements when the next generation inter-Alu sequencing patterns have the quality of being present in sequence reads in a threshold number of regions.

17. The method of claim 11, further comprising the step of: (f) identifying relevant-inter-Alu-genetic-elements when the next generation inter-Alu sequencing patterns have the quality of being aligned at a threshold level to a first reference sequence.

18. The method of claim 11, further comprising the step of: (f) identifying relevant-inter-Alu-genetic-elements when the next generation inter-Alu sequencing patterns have the quality of being aligned at a threshold level compared with to a second reference sequence.

19. The method of claim 11, further comprising the step of: (f) identifying relevant-inter-Alu-genetic-elements when the next generation inter-Alu sequencing patterns have the quality of being variant-genetic elements that do not have biasing features bases within a threshold number of nucleotides of the variant.

20. The method of claim 11, further comprising the step of associating the relevant-inter-Alu genetic elements with the trait when the next generation inter-Alu sequencing patterns have the quality of being aligned at a threshold value of importance to a homeostasis marker of the trait.

21. The method of claim 11, further comprising the step of associating the relevant-inter-Alu genetic elements with the trait when the next generation inter-Alu sequencing patterns have the quality of being aligned at a threshold value of trait severity.

22. The method of claim 11, further comprising the step of associating the relevant-inter-Alu genetic elements with the trait when the next generation inter-Alu sequencing patterns have the quality of being aligned at a threshold value of an age of onset of the trait.

23. The method of claim 11, further comprising the step of associating the relevant-inter-Alu genetic elements with the trait when the next generation inter-Alu sequencing patterns when the relevant-inter-Alu-genetic elements have the quality of being aligned at a specificity to the trait.

24. The method of claim 11, further comprising the step of selecting the paired set of discovery samples from an individual having cell types with different traits.

25. The method of claim 11, further comprising the step of selecting the paired set of discovery samples from a pedigree with different traits.

26. The method of claim 11, further comprising the step of selecting the paired set of discovery samples from a subset of a single cohort with different traits.

27. The method of claim 11, further comprising the step of selecting the paired set of discovery samples from a subset of a cohort with different traits.

28. The method of claim 11, further comprising the step of selecting relevant-inter-Alu-genetic elements to be a loss of heterozygosity.

29. The method of claim 11, further comprising the step of selecting relevant-inter-Alu-genetic elements to be a somatic indel, wherein the somatic indel is an insertion or deletion of genetic information.

30. The method of claim 11, further comprising the step of selecting relevant-inter-Alu-genetic elements to be a small nucleotide variation (“SNV”).

31. The method of claim 11, further comprising the step of selecting relevant-inter-Alu-genetic elements to be a CpG loci DNA methylation.

32. The method of claim 11, further comprising the step of selecting relevant-inter-Alu-genetic elements to be a frameshift variant.

33. The method of claim 11, further comprising the step of selecting the association of the relevant-inter-Alu element with the relevant-inter-Alu-component phenotype to be identified by a threshold value of the coincidence of the relevant-inter-Alu element and the relevant-inter-Alu component phenotype within a set of discovery samples.

34. The method of claim 11, further comprising the step of selecting the set of discovery samples to include both affected samples and unaffected samples, wherein affected samples are samples associated with the relevant-inter-Alu component phenotype, wherein unaffected samples are samples not associated with the relevant-inter-Alu component phenotype.

35. The method of claim 11, further comprising the step of selecting the trait to be a disease, a phenotype, a quantitative or qualitative trait, a disease outcome, or a disease susceptibility.

36. The method of claim 35, further comprising the step of selecting the trait to be tumorigenic.

37. The method of claim 11, further comprising the step of selecting the relevant-inter-Alu element further to be an element associated with one or more genetic elements associated with the trait.

38. The method of claim 35, further comprising the step of selecting the one or more genetic elements to be derived from DNA sequence data, genetic linkage data, gene expression data, antisense RNA data, microRNA data, proteomic data, or a combination thereof.


SEQUENCE LISTING (10 sequences)

SEQ ID NO: 1 - DNA, 18 bp, Artificial Sequence
  synthesized
gagcgagactccgtctca

SEQ ID NO: 2 - DNA, 21 bp, Artificial Sequence
  synthesized
tggtctcgatctcctgacctc

SEQ ID NO: 3 - DNA, 19 bp, Artificial Sequence
  synthesized
aggctgaggcaggagaatg

SEQ ID NO: 4 - DNA, 11 bp, Artificial Sequence
  synthesized
tttaataaaaa

SEQ ID NO: 5 - DNA, 11 bp, Artificial Sequence
  synthesized
ctataatccca

SEQ ID NO: 6 - DNA, 12 bp, Artificial Sequence
  synthesized
agcgagactccg

SEQ ID NO: 7 - DNA, 16 bp, Artificial Sequence
  synthesized
ggctcaagcgatcctc

SEQ ID NO: 8 - DNA, 16 bp, Artificial Sequence
  synthesized
tatgatcgtgccactg

SEQ ID NO: 9 - DNA, 16 bp, Artificial Sequence
  synthesized
acctcaggtgatccac

SEQ ID NO: 10 - DNA, 16 bp, Artificial Sequence
  synthesized
aacaagagcgaaactc